(19) United States
(12) Patent Application Publication
(10) Pub. No.: US 2023/0386670 A1
(43) Pub. Date: Nov. 30, 2023

(54) METHOD AND SYSTEM FOR RECOMMENDING TREATMENT PLANS, PREVENTIVE ACTIONS, AND PREPAREDNESS ACTIONS

(71) Applicant: EHRsynergy LLC, Winter Garden, FL (US)

(72) Inventors: Edward Makaron, Winter Garden, FL (US); Oleg Muza, Winter Garden, FL (US)

(21) Appl. No.: 18/355,036

(22) Filed: Jul. 19, 2023

Related U.S. Application Data
(63) Continuation of application No. 15/870,152, filed on Jan. 12, 2018.

(51) Int. Cl. G16H 50/20; G16H 10/60; G16H 50/70; G16H 10/20

(52) U.S. Cl. CPC G16H 50/20; G16H 10/60; G16H 50/70; G16H 10/20

(57) ABSTRACT

A method, system and computer readable medium for recommending treatment plans, preventive actions, and preparedness actions is provided in this disclosure. The present disclosure relates to a platform for assisting practitioners during patient encounters to receive, and input patient information. The present disclosure relates to a platform configured to manage, analyze, determine, correlate, sort, categorize, and organize patient information, external data, and other related data. The present disclosure relates to a platform configured to analyze the data from the metrics and data sources to recommend diagnoses, make predictions, make recommendations, make suggestions, and provide further data relating to electronic medical records and related information.

### RELATED APPLICATION

This application is a Continuation of U.S. application Ser. No. 15/870,152 filed on Jan. 12, 2018, which is hereby incorporated by reference herein in its entirety.

It is intended that the above-referenced application may be applicable to the concepts and embodiments disclosed herein, even if such concepts and embodiments are disclosed in the referenced applications with different limitations and configurations and described using different examples and terminology.

### FIELD OF DISCLOSURE

The present disclosure generally relates to electronic medical records. The present disclosure relates to a method and system for recommending treatment plans, preventive actions, and preparedness actions.

### BACKGROUND

In some situations, entry of patient information by users or practitioners is quite cumbersome. For example, entry or recording of basic patient information and extensive documentation of each patient encounter requires vast amounts of written paperwork or multiple entries in data entry systems. Thus, the conventional strategy is to centralize patient data by using electronic medical records and computer based data entry systems to enter patient information and document patient encounters.

This often causes problems because the conventional strategy does not account for reducing the cumbersome nature of the data entry process including, but not limited to, redundant entries, and multiple input selections. For example, prior electronic medical records data entry systems may require a user to cycle through numerous fields of medical diagnoses and treatment plans available for a patient who has a history of visiting the treatment center on multiple occasions for the same reason. Existing systems require users to perform redundant and repeated actions for each tier of data entry of information.

In some situations, real-time analysis of patient data with historical patient data or patient data from patients in the electronic records system who have experienced similar symptoms in a manner that provides actionable information or suggestions to the user or practitioner is not possible without tremendous resources, additional staff, and other cumbersome challenges. For example, a regular patient who comes in experiencing a rare asthma medical condition which was previously resolved using a non-conventional treatment may not automatically be recommended to receive that same treatment based on the existing electronic medical records systems. Thus, the conventional strategy is to integrate various existing electronic medical records systems and train staff on how to properly utilize such systems. This often causes problems because the conventional strategy does not account for the incompatibilities of varying electronic medical records systems or the difficulties in providing real-time analysis of patient data, patient encounter data, patient population data, and external data including, but not limited to, data from medical databases, World Health Organization (WHO) alerts, and other data sources.

Existing systems do not provide efficient and effective data entry methods that simplify the process and reduce the time necessary to input patient data and patient encounter information. Existing systems do not provide for efficient and effective real-time analytics of patient data, encounter information, external data, and other related data in a manner that provides real-time actionable information. Another deficiency with existing systems is the inability to use this real-time analysis to provide alerts and notifications to users, patients, and/or practitioners. A method and system according to the principles of the disclosure addresses these deficiencies and associated problems.

### BRIEF OVERVIEW

A method and system for recommending treatment plans, preventive actions, and preparedness actions may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure relates to a platform for assisting practitioners during patient encounters to receive, and input patient information. The present disclosure relates to a platform configured to manage, analyze, determine, correlate, sort, categorize, and organize patient information, external data, and other related data. The present disclosure relates to a platform configured to analyze the data from the metrics and data sources to recommend diagnoses, make predictions, make recommendations, make suggestions, and provide further data relating to electronic medical records and related information. The present disclosure may be configured to structure and store data so as to allow for expansive data mining and analysis as well as application of Al (artificial intelligence), which would further enhance practitioner's ability to provide superior care. Another aspect of the present disclosure goes beyond merely reducing redundant data entry and improving technical efficiencies to make a practitioner's life a bit easier, but ultimately to vastly improve the outcome of patient's visit and the reduction of medical errors.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1A illustrates a block diagram of an operating environment consistent with the present disclosure;

FIG. 1B illustrates a Venn Diagram illustrating the connections between the various components of the platform;

FIG. 2 illustrates a dashboard 200 in accordance to some embodiments of the present disclosure;

FIG. 3 illustrates an expanded side panel view 300 in accordance to some embodiments of the present disclosure;

FIG. 4 illustrates an encounter view 400 in accordance to some embodiments of the present disclosure;

FIG. 5 illustrates a block diagram of an operating environment consistent with the present disclosure;

FIG. 5 illustrates a History of Reason for Visit input tier in accordance to some embodiments of the present disclosure;

FIG. 6 illustrates a Review of Symptoms (ROS) input tier 615a in accordance to some embodiments of the present disclosure;

FIG. 7 illustrates a physical exam input tier 715a consistent with the present disclosure;

FIG. 8 illustrates an assessment input tier 815a in accordance to some embodiments of the present disclosure;

FIG. 9 illustrates a treatment plan input tier 915a in accordance to some embodiments of the present disclosure;

FIG. 10 illustrates a family and patient history view 1000 in accordance to the embodiments of the present disclosure;

FIG. 11 illustrates an encounter note view 1100 in accordance to some embodiments of the present disclosure;

FIG. 12 illustrates an encounter note view 1100 in accordance to some embodiments of the present disclosure;

FIG. 13A is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 13B is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 14 is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 15 is a block diagram of a system including a computing device for performing the method of the present disclosure in accordance to some embodiments of the present disclosure;

FIG. 16 is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 17 is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure;

FIG. 18A is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure; and

FIG. 18B is a flow chart of a method for providing A METHOD AND SYSTEM FOR RECOMMENDING TREATMENT PLANS, PREVENTIVE ACTIONS, AND PREPAREDNESS ACTIONS in accordance to some embodiments of the present disclosure.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

### I. Platform Overview

Consistent with embodiments of the present disclosure, a METHOD AND SYSTEM FOR RECOMMENDING TREATMENT PLANS, PREVENTIVE ACTIONS, AND PREPAREDNESS ACTIONS may be provided. This overview is provided to introduce a selection of concepts in a simplified form that are further described below. This overview is not intended to identify key features or essential features of the claimed subject matter. Nor is this overview intended to be used to limit the claimed subject matter's scope.

Thus according to aspects, the present disclosure provides methods, systems, and/or techniques for entering, managing, analyzing, organizing, recommending, and providing data and actions relating to electronic medical records. The present disclosure further may enable a user to detect patterns in the data. The disclosure provides for a tiered input system of patient data. The disclosure provides for data being entered using various input methods and input methodologies.

In another aspect, The present disclosure provides a system for assisting practitioners receive, input, manage, analyze, determine, correlate, sort, categorize, organize, diagnose, predict, recommend, suggest, take metrics, and provide data relating to electronic medical records. The disclosure provides for a method of improved patient diagnostics including recommending a treatment or action.

In another aspect, the system may comprise a platform which performs functions associated with at least one of: a server, a user interface module, application modules, and external datasets. The disclosure provides for a method for providing preventive actions to patients.

In another aspect, the disclosure provides for providing preparedness actions to practitioners. In another aspect, the disclosure provides for predictive analysis of electronic medical record data.

Consistent with embodiments of the present disclosure, a system comprising a platform, application modules, a user interface module, a server, a patient tracking module, and external data is presented. The Application modules comprise a favorites module, a recommendation module, a predictive analytics module, and a patient tracking module. The server comprises a computer, a processor, a network connection, a memory, and patient profile data. Consistent with embodiments of the present disclosure, a brief overview of each of the elements, components and/or modules is provided below.

### Favorites Module

The system may recommend commonly/frequently selections made in accordance to inputs. For example, once we load a patient, and input ‘reasons for visit’, the system may recommend subsequent-tier systems and elements based on commonly used elements associated with the reasons for visit. The elements may be reordered and/or highlighted based on their frequency. As additional tier selections are made, the more refined the favorites selections become for sub-subsequent tiers.

### Alerts and Notifications

The system may be in operative communication with Medical Encyclopedias, WHO databases, Food and Drug Administration (FDA) alerts and notifications, and Up-to-Date diagnostics from various other data sources (i.e. weather sensors, air quality sensors, water sensors, or other sources). In this way, data obtained from these sources can be used to help with the data entry/favorites in stage 1, as well as provide alerts and triggers when certain conditions are met when cross-referencing datasets. Example: if patient record data indicates “allergic to pollen” and pollen count data indicates “HIGH”, then trigger an alert to go out to patient and/or doctor.

### Recommended Treatments

The system may provide assistance in the refinement of treatment plans and initiatives. For example, upon seeing reasons for visit and certain other data inputs, the system may suggest that the practitioner ask some diagnostic questions to the patient and input those responses into the system. Based on the responses and the data obtained from external sources, the system may provide recommendations to the practitioner (e.g., “quarantine patient immediately”). (The system can suggest based on the above, as well as live sensor information, as well as trending data that we have created based on analysis of data that we have collected internally and/or referenced externally). As another example, the system may review inputs (e.g., reasons for visit, history) and find that certain new treatments have been proven to be effective for similar inputs, with similar patient profiles.

### Predictive Analytics

The system may continue to reference various resources in order to provide predictive analytics. The predictive analytics may include, but not be limited to, for example:

### a) Patient Facing: Preventive Care

The system may track certain patients based on, for example, but not limited to, Coded records associated with those patients (e.g., asthma). The system may further track data received from external sources (e.g., weather data) to meet certain conditions (e.g., high pollen count). The system may then trigger alerts to said practitioner and/or patients to ensure the patient takes preventive measures to avoid aggravating their condition.

For example, the system may analyze data and determined a trend associated with a patient diagnosed with Asthma has a high probability of incurring a negative reaction when exposed to air quality with a value above a predetermined air quality value, X. Consistent with the present disclosure, when the patient is using a navigation software application (i.e. Apple Maps, Google Maps, etc.) to navigate to a destination, the patient may utilize the function of the present disclosure. For example, if the patient has registered to receive alerts or upload their health information to a software application (i.e. Apple's HealthKit™ etc.), if information regarding their Asthma diagnosis is in contained in the software database, preventative actions can be recommended to prevent the negative reaction with poor air quality X. If there is a high probability of the patient encountering poor air quality above a predetermined value X on the current route of travel according to the navigation software, a preventative action or recommendation that the patient take an alternate route around the affected area. The present disclosure may also provide another preventive action or suggestion to avoid the area.

b) Doctor Facing: Prepare for Certain types Care/Consequences of Analytics

Similarly, the system may use the above-mentioned techniques to predict, for example, how many walk-ins the practitioner may have and/or how many patients my need help/advice today on certain topics. This will help the practitioner plan/prepare for a variety of occurrences and events. The predictive analytics can provide a practitioner with a preparedness action or suggestion as a consequence of the analysis.

Although modules are disclosed with specific functionality, it should be understood that functionality may be shared between modules, with some functions split between modules, while other functions duplicated by the modules. Furthermore, the name of the module should not be construed as limiting upon the functionality of the module. Moreover, each stage in the claim language can be considered independently without the context of the other stages. Each stage may contain language defined in other portions of this specification. Each stage disclosed for one module may be mixed with the operational stages of another module. Each stage can be claimed on its own and/or interchangeably with other stages of other modules. The following claims will detail the operation of each module, and inter-operation between modules.

Various hardware components may be used at the various stages of operations follow the method and computer-readable medium claims. For example, although the methods have been described to be performed by a computing device, it should be understood that, in some embodiments, different operations may be performed by different networked elements in operative communication with the computing device. For example, server 110 and/or computing device 1500 may be employed in the performance of some or all of the stages disclosed with regard to the methods claimed below. Similarly, apparatus 105 may be employed in the performance of some or all of the stages of the methods. As such, apparatus 105 may comprise at least those architectural components as found in computing device 1500.

Although the stages are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the system without altering or deterring from the fundamental scope of the depicted methods and systems disclosed herein.

The following disclose various Aspects of the present disclosure. The various Aspects are not to be construed as patent claims unless the language of the Aspect appears as a patent claim. The Aspects describe various non-limiting embodiments of the present disclosure.

Aspect 1. A computer readable medium comprising, but not limited to, at least one of the following:

an input methodology comprising, but not limited to, at least two stages of assisted data entry; and

an input assistance methodology comprising, but not limited to, at least one of the following:

a favorites module,

a recommendation module,

an analytics module; and

a server in communication with the input methodology and the input assistance methodology.

Aspect 2. The computer-readable medium above, further comprising a set of instructions which when executed are configured to enable a method comprising:
wherein the input methodology further comprises:

providing a first interface for receiving data points associated with a patient encounter, wherein the first interface comprises:
an input tier for receiving data points from a user; and

providing a second interface for assistant with an entry of the data points into the first interface, wherein the second interface comprises:
a plurality of suggested data points made available for user selection, wherein at least one selected suggested data point is configured

to populate a currently selected input tier of the first interface; and receiving at least one data element associated with at least one of the following:

a plurality of patient data, and

at least one data point in a patient encounter note during a patient diagnostic process;

analyzing the data element;
wherein the input assistance methodology further comprises recommending, based on the analysis, at least one of the following:

a diagnostic question,

a recommended treatment, and

a recommended action;

determining, based on the analysis, whether an occurrence of an event may cause an adverse effect on a patient;
predicting, based on the analysis, at least one of the following:

a preventive suggestion, and

a preventive action, and

a preemptive preparedness suggestion, and

a preemptive preparedness action;

providing the patient or practitioner with at least one of the following:

the preventive suggestion, and

the preventive action, and

the preemptive preparedness suggestion, and

the preemptive preparedness action.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. Platform Configuration

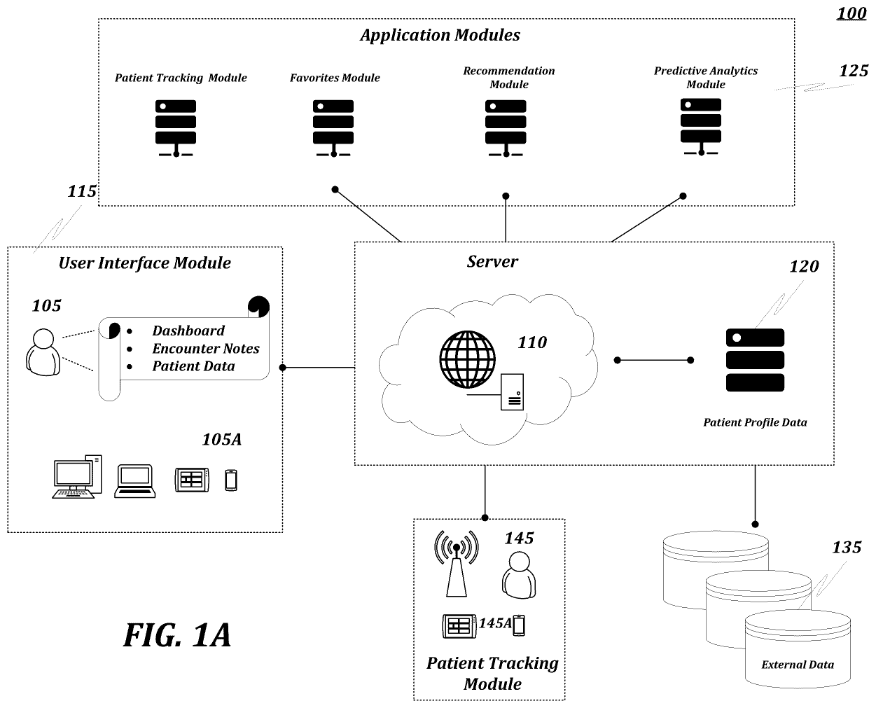

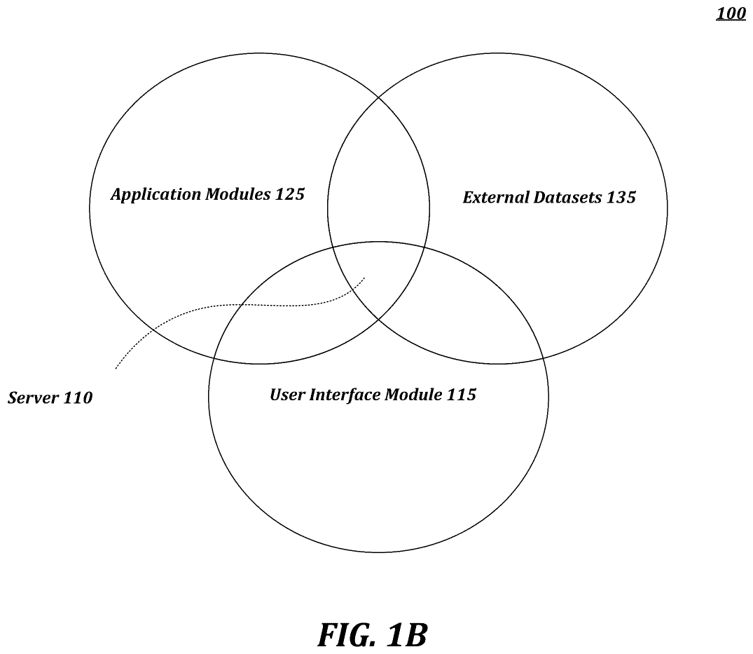

FIGS. 1A and 1B are an illustration of a platform consistent with various embodiments of the present disclosure. By way of non-limiting example, the online platform 100 for managing electronic medical records and recommending treatment plans, preventive actions, and preparedness actions based analysis of patient profile data 120 may be hosted on a centralized server 110, such as, for example, a cloud computing service. The centralized server may communicate with other network entities, such as, for example, a plurality of mobile devices, wearable devices (such as watches or smart glasses), encoding devices, electronic devices (such as desktop computers, laptop computers etc.) and one or more recording devices (e.g., camera drones, handheld camera, or installed cameras) over a communication network, such as, but not limited to, the Internet. Further, users of the platform may include, but are not limited to, practitioners (e.g. physicians), practitioner support staff (e.g. nursing assistant, patient care attendant, medical assistant, aide, medical secretary, home care aide, or other staff), and other users (e.g. patients). Accordingly, electronic devices operated by the practitioner, practitioner support staff, patient, and other users may be in communication with the platform.

A user 105, such as a practitioner, practitioner support staff, patient, or other user, may access platform 100 through a software application. The software application may be embodied as, for example, but not be limited to, a website, a web application, a desktop application, and a mobile application compatible with a computing device 1500.

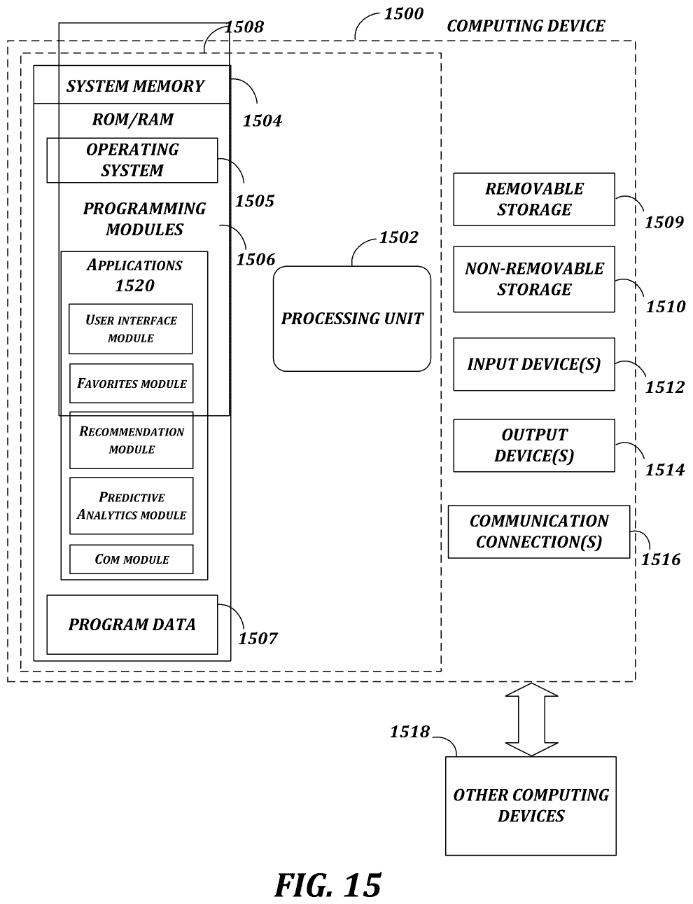

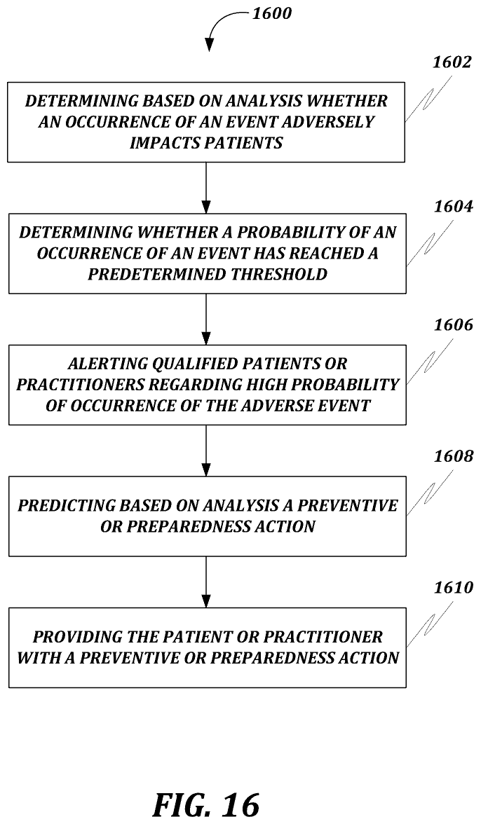

As will be detailed with reference to FIG. 15 below, the computing device through which the platform may be accessed may comprise, but not be limited to, for example, a desktop computer, laptop, a tablet, or mobile telecommunications device. Though the present disclosure is written with reference to a mobile telecommunications device, it should be understood that any computing device may be employed to provide the various embodiments disclosed herein.

The aforementioned modules may be used and interconnected in various combinations with one another. By way of non-limiting example, any of the application modules may be interconnected and/or used in conjunction with at least one of: a user interface module, a patient tracking module, external data or datasets. The application modules comprising at least one of: a favorites module, a recommendation module, a patient tracking module, a predictive analytics module, and a user interface module. The modules may be in communication with a central server. In an embodiment, the modules may reside within a computer readable medium, a server, a computer, a phone, a mobile device, a wearable device, or other electronic device.

In another embodiment, the modules can also be used together in conjunction and/or interconnected as separate elements of a system. In another embodiment, the modules may to be interconnect and/or configured and/or working together as separate elements or components, while not part of or contained within the same component. In other embodiments, the modules may be contained within the same component and/or interconnected within one component or portion of a component and/or components.

In an embodiment, the module may be configured and/or used as an element and/or elements of a system. In yet other embodiments, functionality for any of the aforementioned modules can be interconnected such that the functionality is embodied in one individual module alone or collectively in a portion and or subset of the modules. The modules can be embodied as software, hardware or a combination thereof. The modules can also be collectively embodied in one device, server, computer readable medium, or hardware element.

The following is made with reference to FIGS. 1A and 1B.

I. Embodiments of the present disclosure provide a software and hardware platform comprised of a distributed set of modules, including, but not limited to:

A. User Interface Module 115;

B. Favorites Module 125; and

C. Recommendation Module 125.

In some embodiments, the present disclosure may provide an additional set of modules for further facilitating the software and hardware platform. The additional set of modules may comprise, but not be limited to:

A. Patient Tracking Module 125 and/or 145;

B. Predictive Analytics Module 125; and

C. Communications Module.

In some embodiments, the present disclosure may provide an additional set of sub-modules for further facilitating the software and hardware platform. The additional set of sub-modules may comprise, but not be limited to:

A. Preventive Care Module; and

B. Preparedness Module 125.

FIGS. 1A and 1B illustrate non-limiting examples of operating environments for the aforementioned modules. Although modules are disclosed with specific functionality, it should be understood that functionality may be shared between modules, with some functions split between modules, while other functions duplicated by the modules. Furthermore, the name of the module should not be construed as limiting upon the functionality of the module. Moreover, each stage in the claim language can be considered independently without the context of the other stages. Each stage may contain language defined in other portions of this specification. Each stage disclosed for one module may be mixed with the operational stages of another module. Each stage can be claimed on its own and/or interchangeably with other stages of other modules.

FIG. 1A Description: Still consistent with embodiments of the present disclosure, platform 100 may be configured to access data from various external databases 135. External databases 135 base comprise, but not be limited to, for example medical resources. The data may be analyzed by platform 100 in order to facilitate a plurality of functions and features within platform 100.

FIG. 1B shows a Venn Diagram illustrating the connections between the various components of platform 100. As can be deduced, UI module 115 may have display to user 105 to external datasets for a real time view of the data being communicated to platform 100. Application Modules 125 may further access external datasets 135 for real time, actionable data. UI module 115 may be further enabled to receive updates via Application Modules 125. In some embodiments, server 110 may be configured as an intermediary between each platform component, while in other embodiments, each platform component may communicate directly, without server 110 as an intermediary.

II. Embodiments of the present disclosure provide a software and hardware platform comprised of a distributed set of computing elements, including, but not limited to:

A. A Computing Device

Wherein the platform is operative to control a computing device in furtherance of the operation of the application modules,
The computing device comprising, but not limited to at least one of the following:
   A processing unit,
   A memory storage,
Wherein the computing device may be embodied as a mobile computing device,
   wherein the mobile computing device comprises, but is not limited to,
      A tablet (105A),
      A smartphone (105A),
      A drone,
      A wearable camera (105A),
      A handheld camera (105A),
      An installed camera (105A), and
      A remotely operable recording device;
         Wherein the computing device is configured to perform a method comprising:

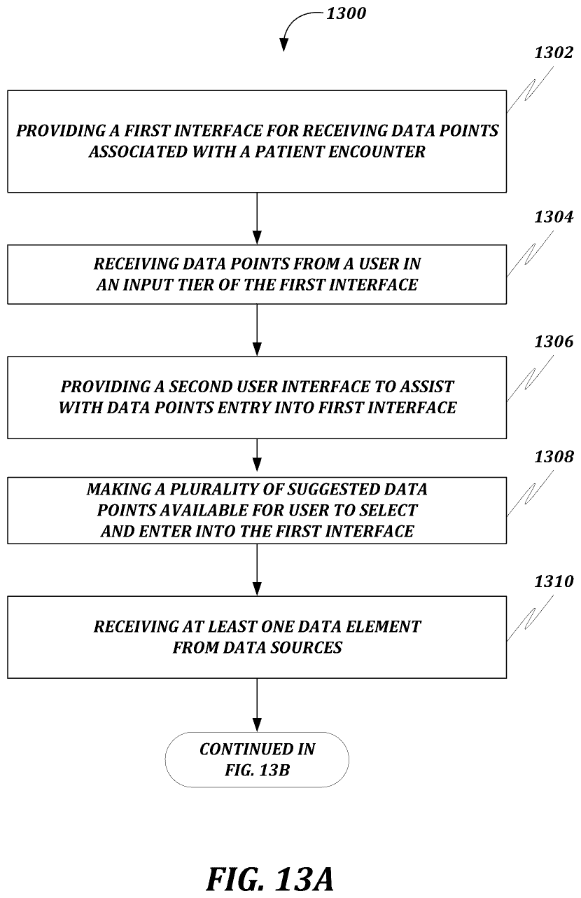

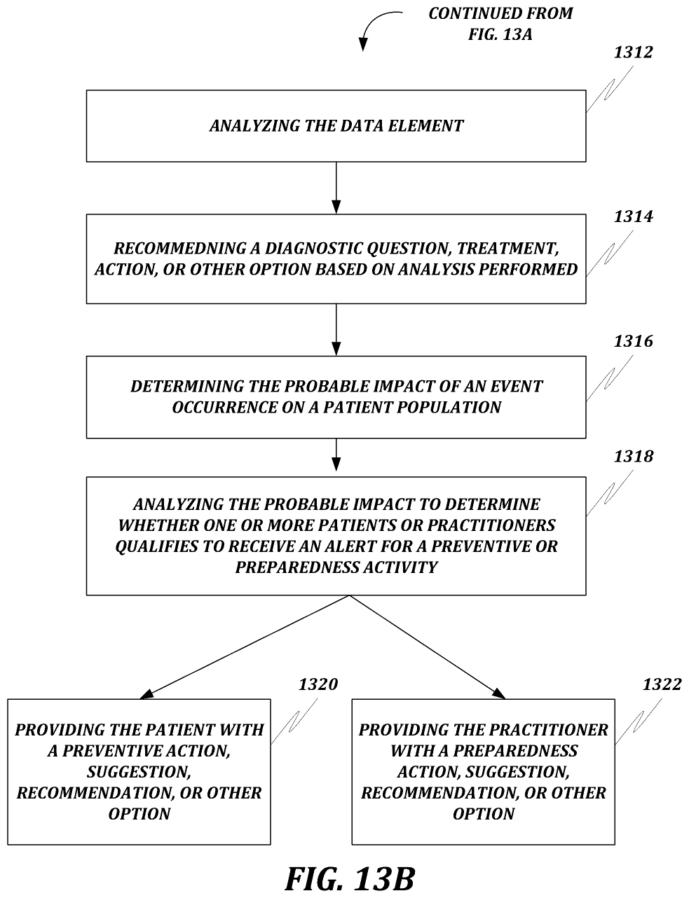

an input method comprising: (FIG. 13A, 1300)
providing a first interface for receiving data points associated with a patient encounter (FIG. 13A, 1302), wherein the first interface comprises:
   an input tier for receiving data points from a user (FIG. 13A, 1304); and
providing a second interface for assistant with an entry of the data points into the first interface (FIG. 13A, 1306), wherein the second interface comprises:
a plurality of suggested data points made available for user selection, wherein at least one selected suggested data point is configured to populate a currently selected input tier of the first interface (FIG. 13A, 1308); and
an improved patient diagnostics method comprising:
receiving at least one data element associated with at least one of the following:
   a plurality of patient data, and
   at least one data point in a patient encounter note during a patient diagnostic process (FIG. 13A, 1310);
   analyzing the data element (FIG. 13B, 1312);

an input assistance method comprising:
recommending, based on the analysis, at least one of the following:
   a diagnostic question,
   a recommended treatment, and
   a recommended action (FIG. 13B, 1314);
      an analytics method comprising:

determining, based on the analysis, whether an occurrence of an event may cause an adverse effect on a patient (FIG. 13B, 1316);
predicting, based on the analysis, at least one of the following:
   a preventive suggestion, and
   a preventive action, and
   a preemptive preparedness suggestion, and
   a preemptive preparedness action (FIG. 13B, 1318);
      an action generating method comprising:

providing the patient or practitioner with at least one of the following:
the preventive suggestion, and
the preventive action (FIG. 13B, 1320), and
the preemptive preparedness suggestion, and
the preemptive preparedness action (FIG. 13B, 1322).

Wherein the computing device may be embodied as any of the computing elements illustrated in FIG. 1A, including, but not limited to, Application Modules 125, Patient Tracking Module 145, User Interface Module 115; and Server 110.

Various hardware components may be used at the various stages of operations follow the method and computer-readable medium. For example, although the methods have been described to be performed by a computing device, it should be understood that, in some embodiments, different operations may be performed by different networked elements in operative communication with the computing device. For example, server 110 and/or computing device 1500 may be employed in the performance of some or all of the stages disclosed with regard to the methods below.

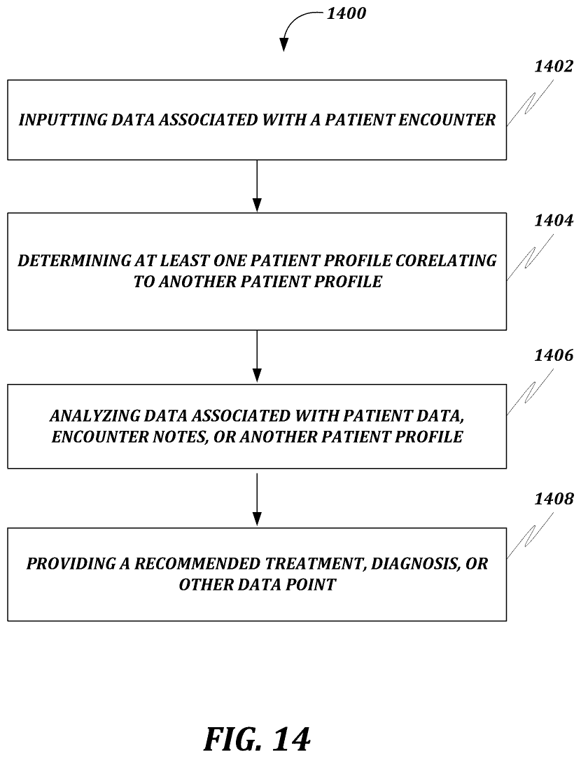

A. Sub-Modules Associated with the Computing Device
Platform may be operative to control at least one of the following sub-modules of a computing device:
   A user interface module,
   A recommendation module,
   A favorites module,
   A predictive analytics module,
   An patient tracking module,
   A preventative care module, and
   A preparedness module.
1. The User Interface Module
   a. Enables user-control of the Computing Device
   b. Enables user-control of the Sub-Modules of the Computing Device:
      i. The user interface module
      ii. The recommendation module
      iii. The favorites module
      iv. The patient tracking module
      v. The predictive analytics module
      vi. The preventative care module
      vii. The preparedness module
      viii. Communications module
   c. Enables user-control of the Platform Modules:
      i. The recommendation module
      ii. The favorites module
      iii. The patient tracking module
      iv. The predictive analytics module
      v. The preventative care module
      vi. The preparedness module
   d. Enables operative control of input medical information hardware during a patient encounter:
      i. Input Device (FIG. 14, 1402)
         1. Keyboard
         2. Mouse
         3. Touch Screen/Touch Pad
   e. Enables capturing based on data:
      i. Recordation of content received from the communications module connected to Input Device
      ii. Recordation of media captured during patient encounter on the computing device (e.g., microphone, audio, video recording)
2. The Favorites Module
   a. Enables Analysis of frequency of captured and stored content:
      i. Enables Frequency Mapping based on, but not limited to, patient data and temporal parameters
      ii. Enables sorting of patient data to determine correlations and frequency. (FIG. 14, 1404)
3. The Patient Tracking Module
   a. Operative control of a location associated with the computing device
   b. In operative communication with a central server, GPS, or location based sensors
   c. Time stamps and location coordinates captured by the patient tracking module
   d. Used for syncing and analysis from various data sources
   e. Enables the reading and communicating of location data associated with a sensing device
   f. The location data may be obtained by way of, for example, but not limited to:
   i. GPS/IP Address/Triangulation
   ii. LAN/WAN
4. The Recommendation Module
   a. Enables the refinement of treatment plans, initiatives, and other suggested courses of action based on analysis of data sources
   b. More effective as more relevant and correlated patient data is made available by data sources (FIG. 14, 1408)
5. The Predictive Analysis Module
   a. Enables predictive analysis for patient preventive care
   b. Enables predictive analysis for practitioner preparedness
   c. The Preventive Care Module
   d. The Preparedness Module
6. The Communications Module
   a. Enables the networking of the multiple application modules associated with multiple networked devices or singular communication device or server
   b. In operative communication with other communications modules of computing devices
   c. Configured to communicate with nearby devices also running on the platform
   d. Configured to join ‘groups’ of devices analyzing data under a similar location, theme, or other type of association
   e. Remote control of the capturing modules
   f. Remote control of the camera
   g. Remote control of the microphone
   h. Via Wireless Media
   i. Via Wired Media
      III. Embodiments of the present disclosure provide a hardware and software platform operative by a set of methods and computer-readable media comprising instructions configured to operate the aforementioned modules and computing elements in accordance with the methods.

The methods and computer-readable media may comprise a set of instructions which when executed are configured to enable a method for inter-operating at least one of the following modules:

I. User Interface Module

II. Favorites Module

III. Recommendation Module

IV. Predictive Analytics Module
a. Preventative Care Sub-Module
b. Preparedness Sub-Module

The aforementioned modules may be inter-operated to perform a method comprising the following stages:
A User Interface Module comprising:
at least two stages of assisted data entry, the input system comprising:
a first stage of input for receiving data points from a patient;
a second stage of input for receiving treatment notes for the patient, and
wherein the first stage of input comprises a plurality of tiers for specifying the data points for the patient, the plurality of tiers comprising at least one of the following:
a first tier for specifying at least one of the following: reasons for visit/chief complaints;
   a second tier for specifying at least one of the following: History of Reason for visit/History of Present Illness (HPI);
   a third tier for specifying a body system associated with at least one of the following: the first tier and the second tier;
   a fourth tier for specifying a condition associated with the body system specified in the third tier;
wherein the second stage of input comprises a plurality of tiers for specifying the treatment notes for the patient, the plurality of tiers comprising at least one of the following:
   a fifth tier for specifying a treatment plan; and
   a sixth tier for specifying a treatment in accordance to the treatment plan.

Variation: Wherein the first stage of input and the second stage of input are compiled into a patient encounter note.

Variation: Wherein a completion of the patient encounter note is facilitated, at least in part, by at least one of the following:
a. A favorites module;
b. A recommendation module; and
c. An analytics module.

A Favorites Module comprising:

providing a first interface for receiving data points associated with a patient encounter, wherein the first interface comprises:
an input tier for receiving data points from a user; and

providing a second interface for assistant with an entry of the data points into the first interface, wherein the second interface comprises:

a plurality of suggested data points made available for user selection, wherein at least one selected suggested data point is configured to populate a currently selected input tier of the first interface; and

A Recommendation Module comprising:

receiving at least one data element associated with at least one of the following:
a plurality of patient data, and
at least one data point in a patient encounter note during a patient diagnostic process;

analyzing the data element

recommending, based on the analysis, at least one of the following:
a diagnostic question,
a recommended treatment, and
a recommended action

Variation: wherein the patient diagnostic process is configured to guide a user through inputting data associated with a patient encounter, and

wherein a recommendation is provided in furtherance of completing the patient diagnostic process;

Variation: determining at least one patient profile similar to the patient profile associated with the patient encounter note;

analyzing data points within at least one patient encounter note associated with the at least one similar patient profile; and

determining a recommendation based on the analysis of the patient encounter note and the at least one patient encounter note associated with the at least one similar patient profile.

Variation: wherein analyzing data points within the at least one patient encounter note associated with the at least one similar patient profile comprises analyzing at least one of the following:

previous encounters for each patient profile;

previous diagnosis for each patient profile; and

previous treatments for each patient profile.

A Predictive Analytics Module comprising:

determining, based on the analysis, whether an occurrence of an event may cause an adverse effect on a patient;

predicting, based on the analysis, at least one of the following:
a preventive suggestion, and
a preventive action, and
a preemptive preparedness suggestion, and
a preemptive preparedness action;
   an action generating method comprising:

providing the patient or practitioner with at least one of the following:
the preventive suggestion, and
the preventive action, and
the preemptive preparedness suggestion, and
the preemptive preparedness action.

Variation: receiving at least one data element associated with at least one of:
a plurality of patient data,
a plurality of external data,
a plurality of patient profile data,
a plurality of encounter notes,
a plurality of patient tracking data,
   other data;
analyzing patient data for a plurality of patients; and
determining whether one or more practitioners qualifies to receive an alert; and
providing an alert to the one or more practitioners.

Variation: determining whether one or more patients qualifies to receive an alert; and
providing an alert to the one or more patients.

Variation: wherein analyzing further comprises:
determining at least one patient profile similar to the patient profile associated with a set of conditions;
analyzing data points within at least one patient data element associated with at least one determined set of conditions; and
determining a preemptive preparedness action based on the analysis of the patient data element and the at least one external data element associated with at least one condition.

Variation: further comprising:
wherein determining further comprises determining whether there is a high probability that a known set of conditions will adversely impact one or more patients; and
wherein analyzing further comprises determining whether a probability of an occurrence an event has reached a predetermined threshold; and
wherein determining further comprises a positive correlation between an external patient data element meeting the predetermined threshold and a patient data element triggers an alert.

Variation: further comprising:
wherein the occurrence of an event is at least one of: high pollen count, poor air quality, high smog level, high lead level in public water supply, poisonous gas released, high radiation levels, terror attack, fire, flooding, earthquake, tornado, heat wave, drought, am FDA issued warning against a drug that is used by the patient, a possible food contamination (e.g. salmonella warning, etc.), or other emergent event.

Although the stages are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the without altering or deterring from the fundamental scope of the depicted methods and systems disclosed herein.

IV. Embodiments of the present disclosure provide a hardware and software platform operative as a distributed system of modules and computing elements.

### User Interface Module

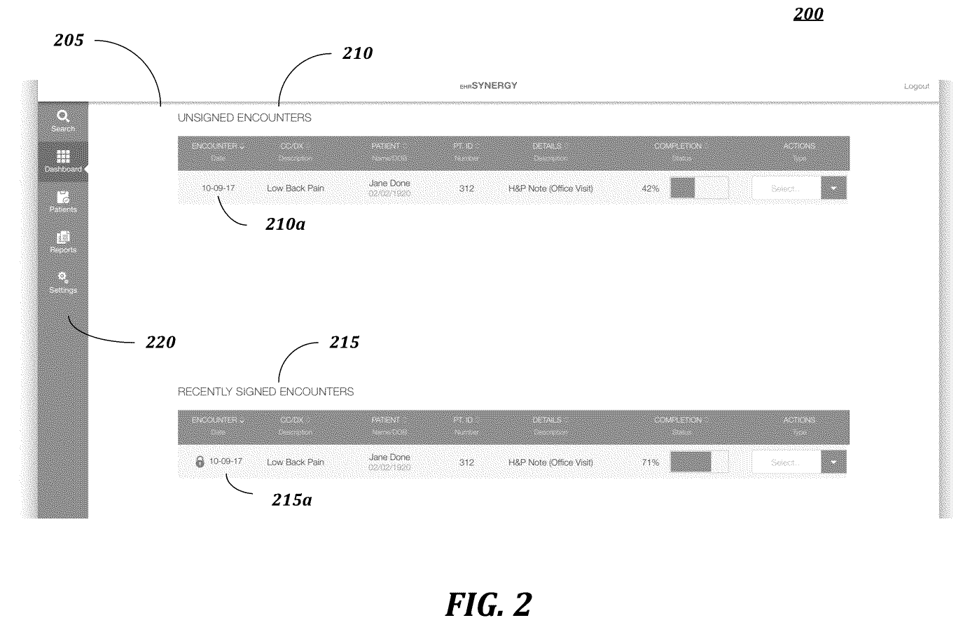

FIG. 2 illustrates a dashboard 200 in accordance to some embodiments of the present disclosure. Dashboard 200 may comprise a main segment 205 and a side panel 220. Side panel 220 may persist throughout the various views of platform 100, and enable user navigation to different platform segments. Main segment 205 may comprise data associated with the dashboard 200, as selected in side panel 230. In some embodiments, dashboard 200 may display patient visit data, which may be referred to as ‘encounters’. Encounters may be segmented by an unsigned encounters view 210 (e.g., incomplete encounters or drafts), comprising line-item encounters 210a, and a recently signed encounters view 215, comprising line-item encounters 210b. Once an encounter note is complete, it may be signed and locked from editing by a provider.

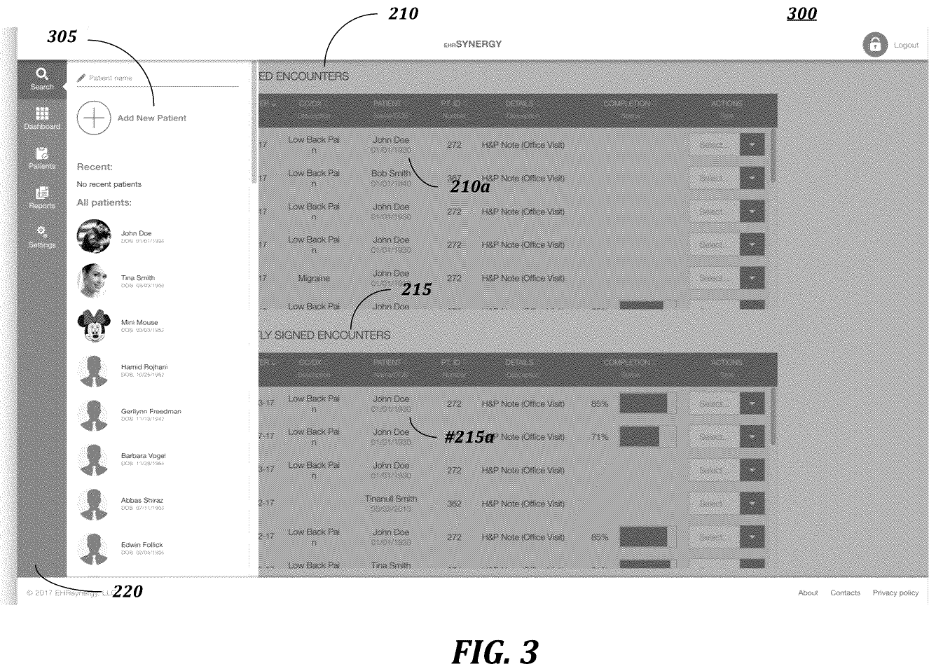

FIG. 3 illustrates an expanded side panel view 300 in accordance to some embodiments of the present disclosure. Side panel view 300 may be triggered upon, for example, a selection of a selectable button in side panel 220. The selectable button may provide an expanded set of options extending from side panel 220, including, but not limited to, for example, adding a new patient selector 305, which, upon selection, may enable the entry of new patent data into platform 100. In some embodiments, recent patient data may be displayed in the expanded side panel 300, as well as a full listing of patient data.

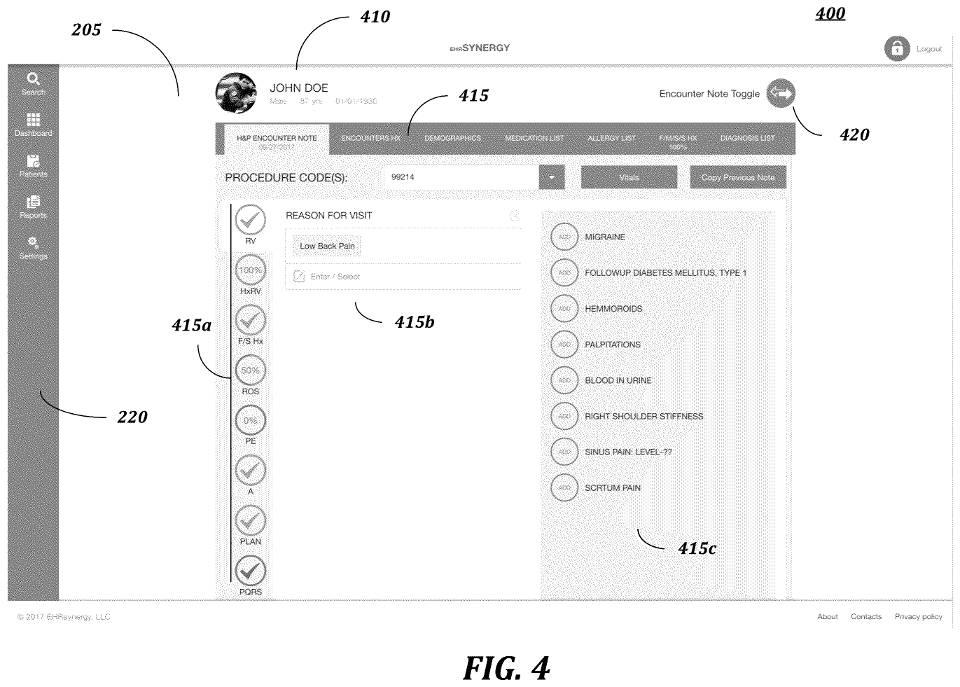

FIG. 4 illustrates an encounter view 400 in accordance to some embodiments of the present disclosure. Encounter view 400 may be presented within main segment 205. An encounter may be related to a patient profile 410. A full profile view may be toggled by encounter note toggle 420. As such, a patient may be associated with a plurality of encounters and platform 100 may be configured to cross reference the plurality of encounters to derive data for the various embodiments disclosed herein. Encounter view 400 may comprise a plurality of tabs 415. Each tab may enable a user 105 to specify a plurality of encounter data. For example, the encounter history tab may display encounter history data for a selected patient, the demographics tab may display patient profile data (e.g., race/ethnicity, and the like), the medication list and allergy list tab may specific active and inactive medications and allergies, respectively.

In some tabs, main segment 205 may further be sub-divided by a input tier panel 415a, which may illustrate the corresponding input tiers for each stage of data entry. FIG. 4 illustrates the specification of, for example, reason for visit input tier 415b. User 105 may select from favorites module view 415c, or favorites module 125. In accordance to the various embodiments herein, each input tier may comprise a favorites module view 415c, which is associated with the underlying processing of the favorites module 125. Thus, the favorites module view 415c may list suggested, frequent, or recommended inputs for each selected tier by invoking the functionality of the favorites module 125. Details with regard to favorites module 125 functionality are disclosed in the subsequent sections of the present disclosure.

As the user completes each input tier, a percentage indicator may appear within the input tier panel, indicating to user 105 whether or not they have completed the necessary inputs for the corresponding tier. Once complete, a complete indicator may appear such as, for example, but not limited to, a check mark. Employing favorites module view 415c enables user 105 to more efficiently and effectively input data, as it provides intelligent suggestions to augment the input process for this tier.

In the illustrated example, user 105 begins specifying the encounter by inputting John Doe's Reason for Visit into the input tier 415a, and is assisted with suggested input items by favorites module view 415c. In some embodiments, the reasons for visit may be referred to as “Chief Complaints.” Further still, and as will be detailed below, certain indicators may appear complete upon the creation of the encounter note, as the data used to complete the corresponding inputs may be retrieved from, for example, a pre-populated patent profile.

The system may be configured to enter the reasons for visit in the patient's own words instead of just the results of the physical exam based on the physician or practitioner.

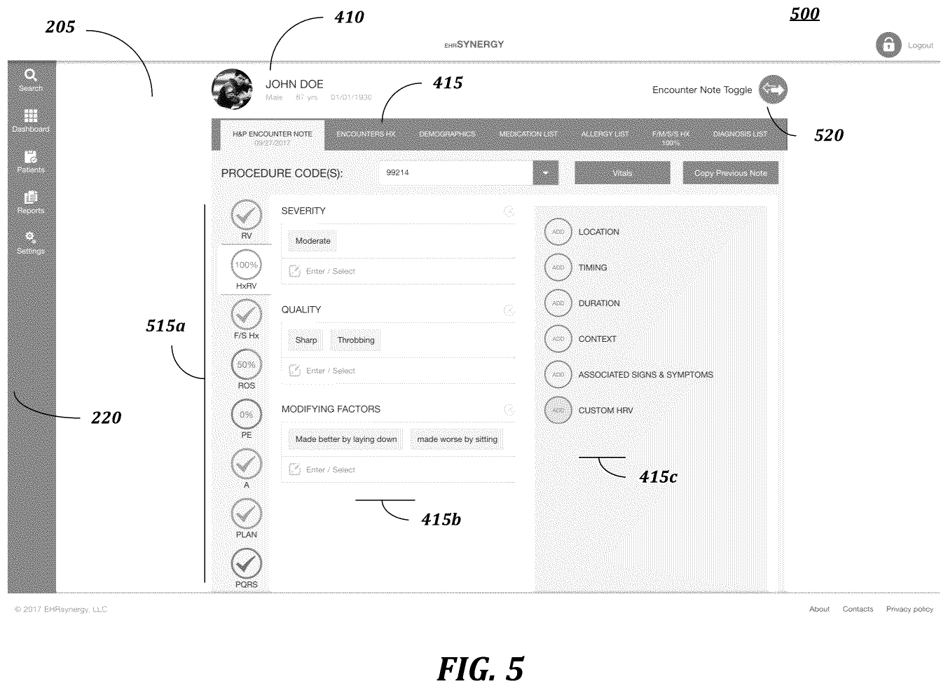

Turning now to FIG. 5, a History of Reason for Visit input tier 515a is illustrated within encounter input view 500. Continuing the illustrated Example, user 105 may now proceed, via subsequent selection in the input tier panel 515a, to inputting John Doe's symptom history as it relates to the reason for visit (may also be referred to as History of Present Illness (HPI) in some embodiments) as illustrated in view 500.

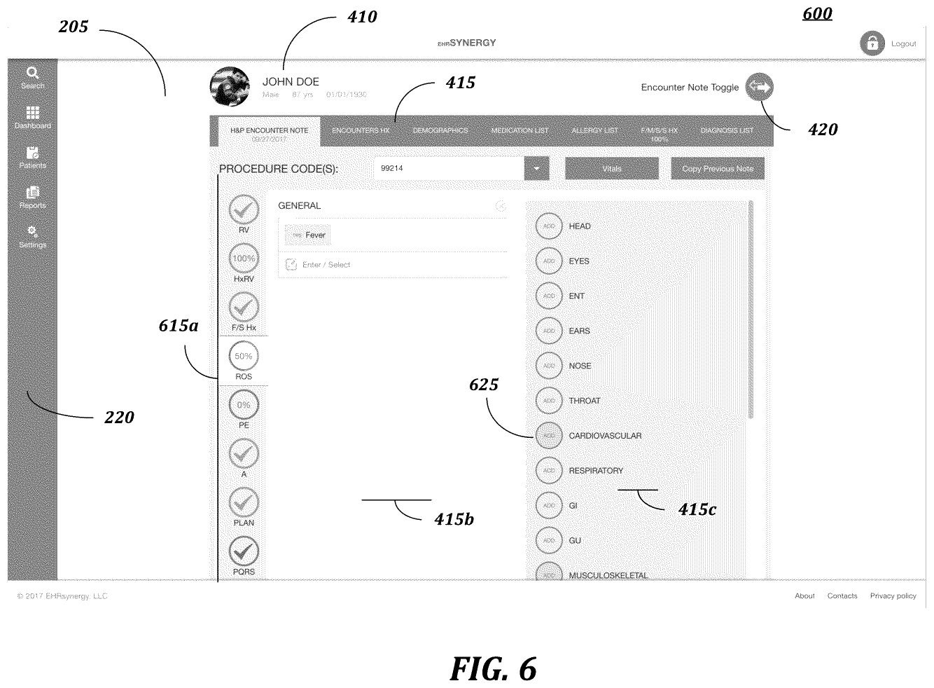

FIG. 6 illustrates a Review of Symptoms (ROS) input tier 615a in accordance to some embodiments of the present disclosure. Continuing the illustrated Example, user 105 may now proceed, via subsequent selection in the input tier panel 515a, to inputting John Doe's symptoms as it is determined by the examiner as illustrated in view 600.

Referring still to FIG. 6, favorites module view 415a may provide user 105 not only with a list of symptoms that may be selected, but also with color coded indicators 625, indicating the likelihood and/or frequency with which the suggested symptom might occur as they relate to the other inputs in the encounter note and related encounter notes. The color coded elements may be blue, yellow, or green indicating varying frequencies. Other colors may be used for other indications such as red, orange, and other colors.

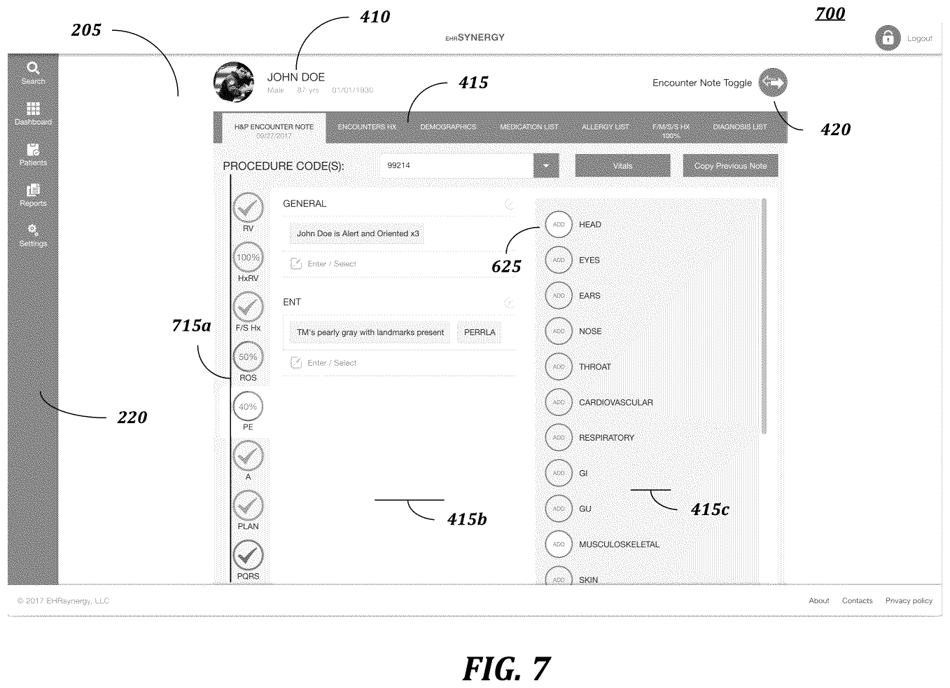

Turning now to FIG. 7, a physical exam input tier 715a may be provided in various embodiments of the present disclosure. Continuing the illustrated Example, user 105 may now proceed, via subsequent selection in the input tier panel 715a, to inputting John Doe's symptoms as it is determined by the examiner as illustrated in view 700. Whereas ROS may be based on the subjective responses of a patient, the PE may be based on a physical exam by a provider.

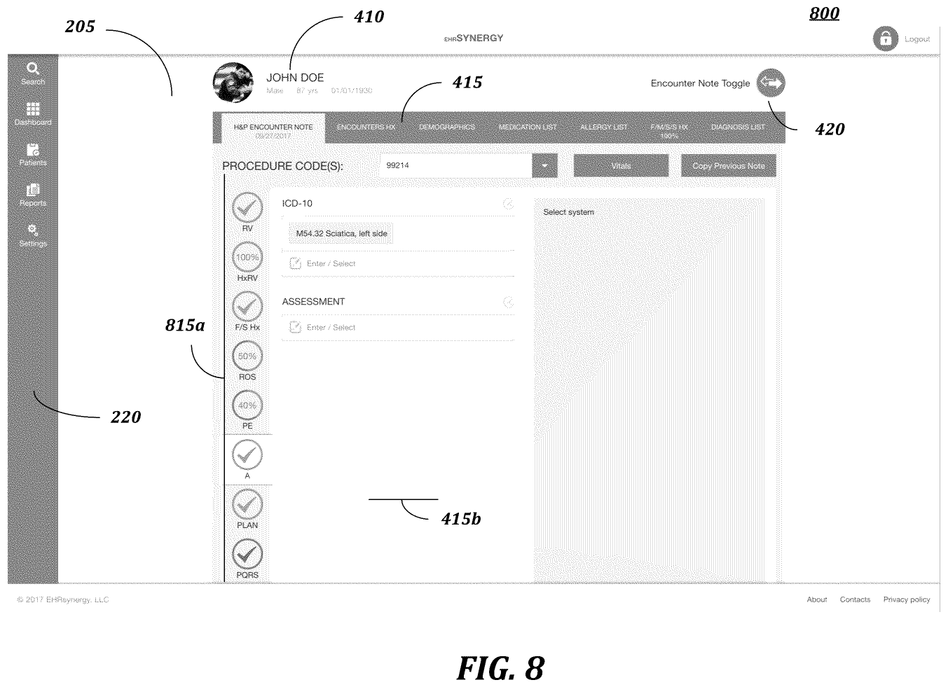

FIG. 8 illustrates an assessment input tier 815a in accordance to some embodiments of the present disclosure. Continuing the illustrated Example, user 105 may now proceed, via subsequent selection in the input tier panel 815a, to inputting an assessment for John Doe's as it is determined by the examiner as illustrated in view 800.

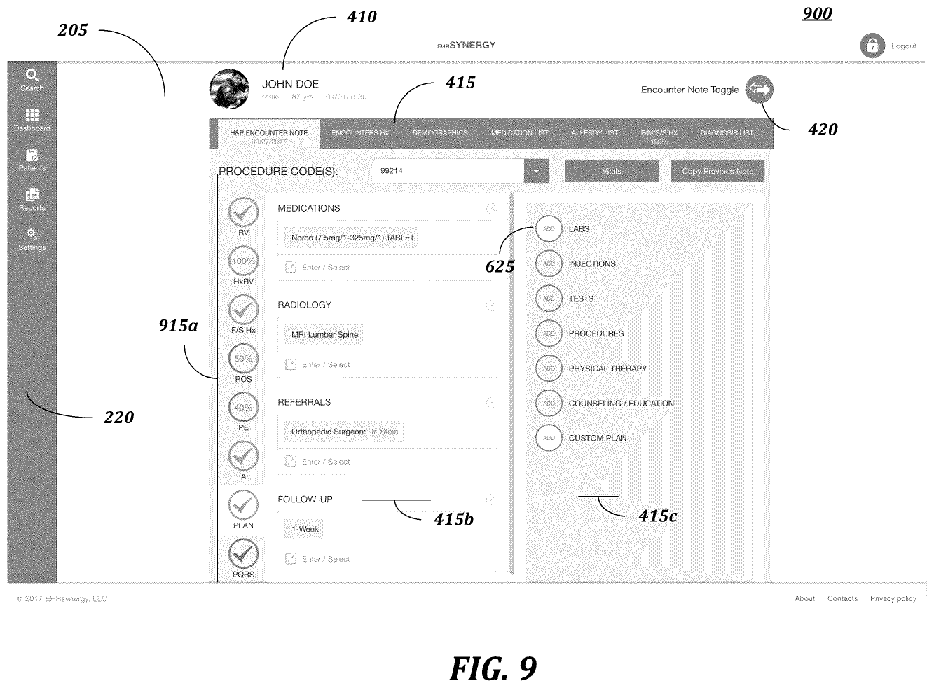

Having inputted an assessment, platform 100 may enable user 105 to input a treatment plan. FIG. 9 illustrates an treatment plan input tier 915a in accordance to some embodiments of the present disclosure. Continuing the illustrated Example, user 105 may now proceed, via subsequent selection in the input tier panel 915a, to inputting a treatment for John Doe's as it is determined by the examiner as illustrated in view 900.

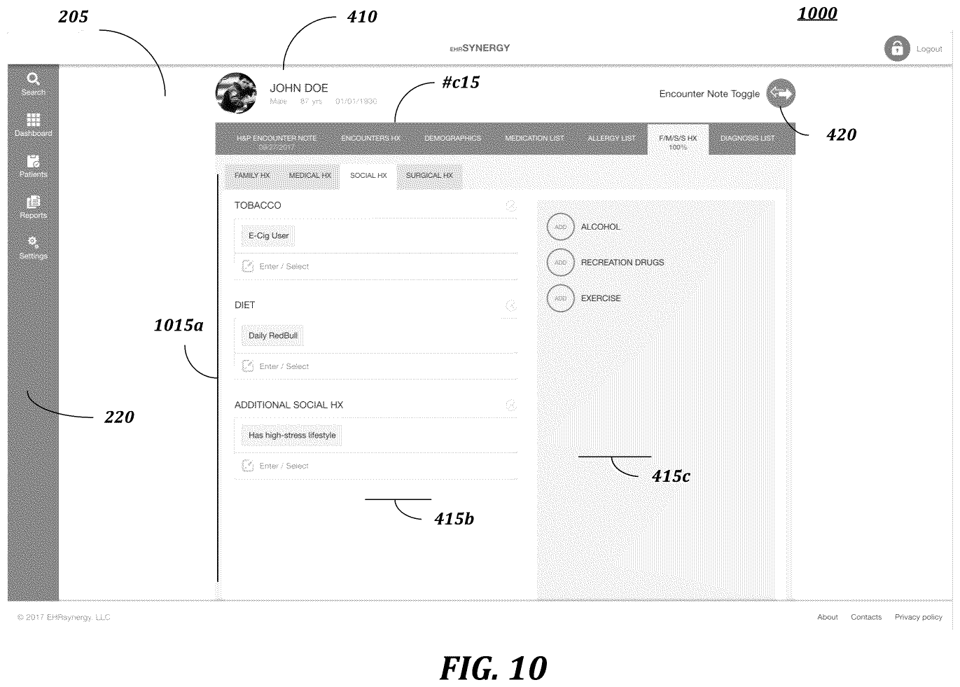

FIG. 10 illustrates a family and patient history view 1000 in accordance to the embodiments of the present disclosure. View 1000 provides user 105 with data points or data elements associated with the patient at hand, their family history, medical history, social history, and surgical history. These data points, data, or data elements may be used when creating a new encounter for the patient. Thus, data from the family and personal history view 1015a may be inputted and pulled in to subsequent encounter notes for the patient. Moreover, the data points may remain up-to-date with every new encounter for the patient, as well as related patients in order to maintain an accurate family history view.

Unlike the other input tiers that specify data for a particular encounter, family and patient history data may be kept consistent across all encounters, as well as the other elements of tabs 415 such as, for example, but not limited to, encounter history, demographics, medication list, allergy list, and diagnosis list. In the illustrated example, user 105 begins specifying the encounter by inputting historical data into the input tier 1015a, and is assisted with suggested input items by favorites module view 415c. The inputted data may, in turn, persist across all encounters associated with John Doe. Accordingly, upon the creation of the encounter, a completion indicator may appear for data which has already been pulled from a pre-existing entry.

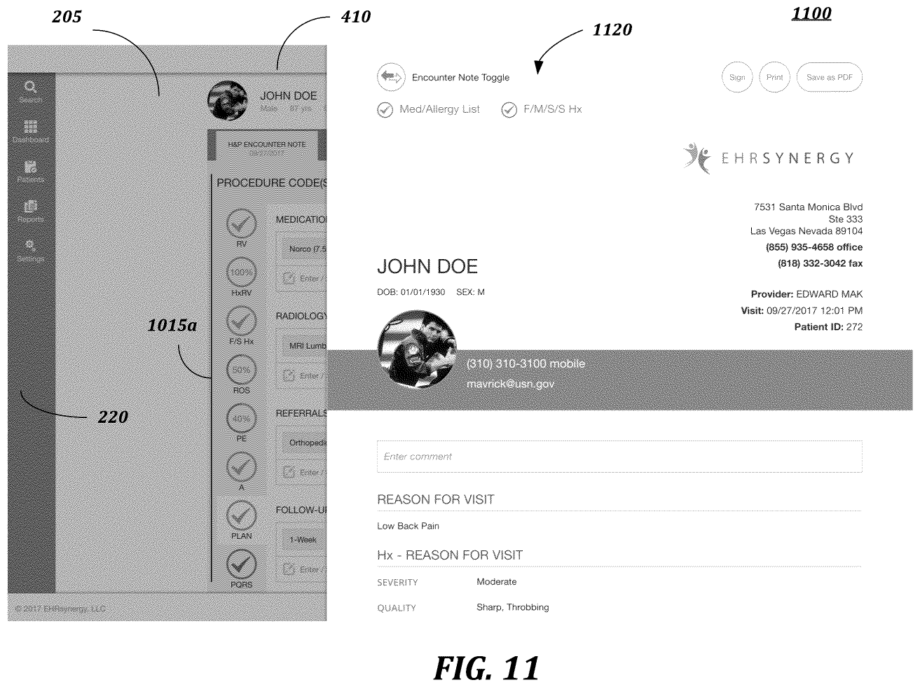

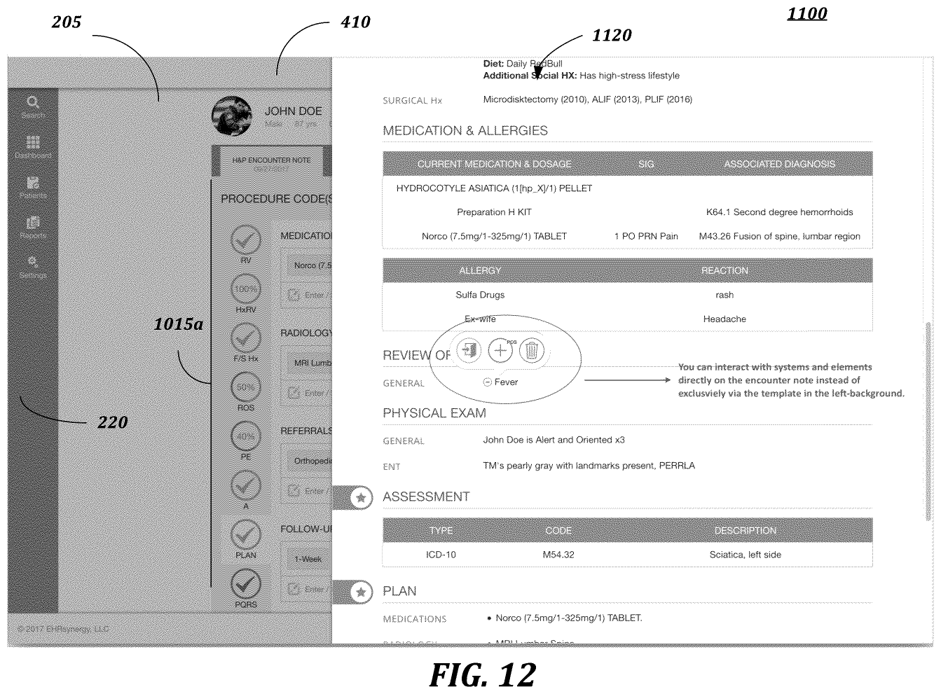

FIGS. 11 and 12 illustrate an encounter note view 1100 in accordance to some embodiments of the present disclosure. Appendix A provides a full view of an example of an encounter note. Encounter note view 1100 can be engaged by user 105's selection of the encounter note toggle 420. Encounter note 1100 provides an overview 1120 of the inputs made and retrieved throughout the various input tiers. However, unlike the main segment view 410, overview 1120 enables the user to review and edit the various data points within the encounter note without the rigidity of the guided template-like inputs of the main segment view 410. The toggle note may further provide functionality to edit any input data, copy data from a previous encounter, or delete data. For example, in 12, the first button can navigate a user to that specific tier. The second button can edit and/or modify the encounter note. The third button may delete the encounter note. In 12, a user may copy a previous visit, previous encounter note, or other data entry point.

### Favorites Module

Embodiments of the present disclosure may provide a module for displaying, for user selection, standardized inputs that are frequently inputted for a corresponding input tier. These displayed inputs made for user selection may be referred to as ‘favorites’ and made available to the user in a ‘favorites view’ or ‘favorites module’ that accompanies the input tiers.

For example, where user 105 loads a patient encounter note and enters a first input tier, such as the ‘reasons for visit’ input tier, the favorites module may be configured to recommend data point, data, or data elements to be used for inputs. As will be detailed below, the recommended data points, data, or data elements may be, for example, previously used data points, data, or data elements. The user may simply select one or more of the recommended data points, from the favorites module, and the selected data points will be populated into the corresponding input tier.

First Type: Helps with Data Point Categories Within an Input Tier

In yet further embodiments of the present disclosure, the favorites module may be configured to assist the user in inputting commonly used data point, data, or data elements categories. Referring to FIG. 5 illustrates one example, wherein user 105 is within the history of reasons for visit' input tier. Some common data point categories that user 105 may enter with regard to this input tier may include, for example, severity, quality, and modifying factors.

As user 105 inputs the data points associated with such categories, favorites module 415c may further suggest to the user additional data point, data, or data elements categories that may be valuable for user 105 to further specify, such as, for example, but not limited to: location, timing, duration, context, and associated signs & symptoms. In this way, as user 105 completes the patient encounter note, favorites module 415c may be configured so as to enable user 105 to quickly and effectively ensure that the input tier is fully and sufficiently specified.

FIG. 9 illustrates yet another example of favorites module 415c providing recommended categories of input tiers. In this instance, favorites module 415c may provide recommendations of data point, data, or data elements categories in a second stage of input tiers, in which the treatment portion of the patient's encounter note is specified.

Favorites module 125 may be enabled to determine the categories of data points for a given input tier based on, for example, but not limited to, historical data. In some embodiments, favorites module 125 may be configured to analyze the various categories of data points that various users of platform 100 have entered for various patient encounter notes. The analysis may then determine which data point categories are frequently used in conjunction with corresponding input tiers. In this way, favorites module 415c may be configured to comprise frequently used data point categories for each input tier.

Second Type: Helps With Actual Data Points Within the Input Tier

Embodiments of the present disclosure may enable a plurality of methods for determining favorite data points, data, or data elements made available for user selection in the favorites module. For example, some data points made for selection in the favorites module may be based on those data points that have already been entered by the user in previous input tiers or a currently selected input tier.

Referring to FIG. 6, wherein user 105 is completing an input tier for specifying a body system associated with a patient's symptoms, favorites module 415c may display standardized inputs for body systems commonly entered along with the inputs made in the completed input tiers. Accordingly, favorites module 415c may be configured to analyze user inputs made in completed input tiers (e.g., the ‘reasons for visit’ input tier) and analyze one or more internal and/or external datasets in order to determine those body systems that frequently accompany the inputs made in completed tiers.

### Favorites Module Analysis

Consistent with embodiments of the present disclosure, favorites module 125 may be configured to suggest data points (e.g., elements) or data point categories (e.g., systems) in accordance to a plurality of methods, and through a plurality of systems. Some of those methods and systems include, but are not limited to, for example:

Analysis performed on previous inputs for a current patient encounter note;
Analysis performed on previous patient encounter notes for the same patient;
Analysis performed on previous patient encounter notes of a plurality of patients;
Wherein the plurality of patient encounter data is accessed through a dataset of encounter note data internal to platform 100;
Wherein the plurality of patient encounter data is accessed through a dataset of encounter note data external to platform 100.

The aforementioned analyses may be based on, at least in part, a determination of which data points or data point categories frequently occur together within an encounter note and/or within an input tier of an encounter note. In this way, favorites module 125 may determine which data points or data point categories to display within favorites module 415c.

In some embodiments, data read from external databases 135 may be used to facilitate recommendations in a favorites module 125. In turn, favorites module 125 may have access to patient data from a plurality of different practices in order to, for example, perform its analysis so as to facilitate a determination of data point or data point category frequency. In this way, favorites module may suggest data points, data, or data elements for selection in a first input stage, as well as recommendations for treatment and treatment plans in a second input stage. It should be understood that data may be anonymized and presented in a way so as to be HIPPA compliant.

Still consistent with embodiments of the present disclosure, favorites module 125 may determine a frequency rate that certain data points or data point categories appear in correlation with an encounter note and/or an input tier within an encounter note. This frequency rate may, in turn, be indicated to user 105 through the user interface module. For example, in some embodiments, a color-coded indicator may be displayed in favorites module 415c next to each suggested data point or data point category.

One such example is illustrated in FIG. 6, with reference to element 625. In this example, favorites module 415c provides a color-coded indicator 625 next to each suggested data point. The magnitude or wave-length of the color may be correlated with a frequency with which the data point is used, thereby attracting user 105's attention in selecting the data point. In this way, user 105 may be assisted in efficiently and effectively completing the patient encounter note.

### Recommendation Module

In certain embodiments, platform 100 may comprise a recommendation module. The recommendation module may suggest data points or data point categories for various input tiers of an encounter, in a similar way to the favorites module's operation. In further embodiments, however, the recommendation module may be used to facilitate an improved diagnostic experience for user 105 and a patient that is the subject of the patient encounter. It should be noted that, throughout the various embodiments disclosed herein, recommendation module need not be used in conjunction with other components of platform 100 (e.g., UI module, an encounter note, favorites module, or analytics module). Rather, the disclosed features and functions of the recommendation module may be employed independently of other modules in platform 100.

First Type: Without External Data

Consistent with embodiments of the recommendation module may have access to internal data 120, including, but not limited to, for example, patient data and encounter note data. The recommendation module may employ internal data 120 to further use to assist user 105 in interacting with the patient.

As one example, when analyzing a patient encounter's reasons for visit and other data points entered throughout the various tiers of an encounter note, the recommendation module may suggest that the practitioner ask some diagnostic questions to the patient and input those responses into the system.

As another example, platform 100 may correlate user 105 inputs for one encounter note with those inputs made in other encounters on patients with similar patient profiles where certain new treatments were tried and have been proven to be effective.

In some embodiments, user 105 may be directed to provide additional data points addressing the recommendation module's diagnostic recommendations. These additional data points may then, in turn, be further analyzed by user 105 to, for example, ask additional diagnostic questions, provide recommended treatments, or recommend certain actions. In this way, whereas the favorites module may be used to assist user 105 with data point entry, the recommendation module may be used to user 105 with patient diagnostic and treatment.

Second Type: With External Data

By means of external datasets 135, platform 100 may be in operative communication with, for example, but not limited to, Medical Encyclopedias, WHO databases, and up-to-date diagnostics from various other data sources (i.e. weather sensors, air quality sensors, water sensors, and the like).

The external data analyzed from such external dataset sources can be used to assist user 105 throughout the various input tiers of an encounter note and/or the patient diagnostic process. These additional data points, data, or data elements may then, in turn, be further analyzed to, for example, ask additional diagnostic questions, provide recommended treatments, or recommend user 105 take certain actions.

As on example, based at least in part on data read from external sources, the recommendation module may provide recommended actions to user 105 (e.g., “quarantine patient immediately”). As another example, platform 100 may review inputs (e.g., reasons for visit, history) and find that certain new treatments have been proven to be effective for similar inputs, with similar patient profiles. In turn, these treatments may be recommended to user 105.

### Recommendation Module Analysis

Consistent with embodiments of the present disclosure, recommendation module may be configured determine suggest data points, data, data elements or data point categories in accordance to a plurality of methods, and through a plurality of systems. Some of those methods and systems include, but are not limited to, for example:

Analysis performed on previous inputs for a current patient encounter note;
Analysis performed on previous patient encounter notes for the same patient;
Analysis performed on previous patient encounter notes of a plurality of patients;
Wherein the plurality of patient encounter data is accessed through a dataset of encounter note data internal to platform 100;
Wherein the plurality of patient encounter data is accessed through a dataset of encounter note data external to platform 100.

In certain embodiments, the recommendation module may work in conjunction with favorites module. For instance, whereas the favorites module is used to assist in data point, data, or data elements entry, the recommendation module may be used to perform additional analysis based on external data and provide the favorites module 415a with suggested data points, data, or data elements or data point categories of inputs. In this way, favorites module 415a may now be enabled to derive data points, data, or data elements for suggested entry beyond a mere frequency of data point, data, or data elements occurrence analysis.

### Predictive Analytics Module

Embodiments of the present disclosure may provide an analytics module for making predictions based on an analysis of data accessible to platform 100. The predicative analytics module may be in operative communication with, but not limited to, for example, the patient profile data and the external datasets. The predictive analytics may include, but not be limited to, the following features and functionality.

### Patient Facing: Preventive Care

The predictive analytics module may be configured to track and monitor a plurality of data sources, including, but not limited to profiles stored in a) patient profile database 120, b) external data sets 135, and c) patient tracking data 145. The module may use the data obtained via profile 100 in order to trigger certain actions when the profile data meets the criteria for triggering the actions.

### Patient Profile Data

Consistent with the various embodiments herein, the module may have access to coded records associated with, for example, patient encounters, patient allergies, patient medical history, and the like. In one example, the module may analyze the coded records in a patient profile in order to determine if a profile qualifies for an alert. In other example, the module may analyze the profile data based on, for example, certain characteristics of the profile data. The characteristics may be derived from, for example, the data point entries in the various patient encounter notes within the patient profile data. The process for determining whether the patient profile qualifies for an alert is detailed below.

A) Patient Tracking Data

In yet further embodiments, the predictive analytics module may be in communication with patient monitoring and tracking data, as provided by the patient tracking module 145. The patient tracking data may comprise up-to-date information on, for example, but not limited to, a patient's biometrics, location, destination, and various other telemetric data that may be collected by the patient tracking module 145. Patient tracking module 145 may be configured to monitor and track the data by way of, for example, but not limited to, a computing device associated with the patient and various peripherals thereto.

For example, patient tracking module 145 may be integrated with wearable computing devices configured to track, for example, a patient's heart rate, respiratory rate, coughing, and blood oxygen levels. In this way, patient tracking module 145 may be enabled to receive biometric data associated with a patient. As another example, patient tracking module 145 may be in operative communication with a mobile computing device having a location detection module (e.g. a smartphone). In turn, patient tracking module 145 may associate the tracked and monitored data (i.e., biometrics and location) with the patient profile, thereby supplementing the patient profile data with up-to-date patient data.

B) External Data

Still consistent with the various embodiments herein, the module may receive data from external data sources 135. External data sources 135 may be accessed by the module in order to share data including, for example, weather data, allergen data, and the like. External data sources 135 may also provide information which includes, for example, but is not limited to, Medical Encyclopedias, WHO databases, and up-to-date diagnostics from various other data sources.

### Preventive Care Analysis

Consistent with the embodiments of the present disclosure, the predictive analytics module may be configured to determine and/or suggest a preventive action, a plurality of preventive actions, or a series of suggested preventive actions to be taken by the patient in accordance to a plurality of methods, and through a plurality of systems. Some of those methods and systems include, but are not limited to, for example:

Analysis performed based on a defined set of rules and conditions associated with a plurality of patient data including at least one of: patient profile data, patient tracking data, external data;
Wherein the plurality of patient data further comprises data elements;
Wherein the data elements further comprise at least one of: coded records, patient encounter data, patient monitoring data, external databases, external diagnostic data;
   Analysis performed on data elements based on the rules and conditions;
Wherein rules and conditions include a patient data element;
   Analysis performed on external data to determine whether an element of the external data meets a predetermined threshold;
Wherein the predetermined threshold is associated with the set of conditions;
Wherein the set of conditions may impact one or more patients;
Wherein an positive association match between the external patient data element meeting the predetermined threshold and a patient data element triggers an alert;
   Analysis performed on patient data to determine whether profile qualifies for an alert;
   Analysis performed on patient data for a plurality of patients to determine whether one or more patients qualifies to receive an alert;

The predictive analytics module further provides methods and systems for making predictions based on an analysis of data accessible to platform 100. Platform 100 may monitor data sources in real-time such that the information available to platform 100 is consistently updated with the most up-to-date information.

The predictive analytics module may further be configured to correlate patient data against a certain set of rules and conditions. The module may trigger alerts and notifications as a result of analyzing external data relating to the set of conditions and making the determination that certain conditions are present which may impact specific patients.

By way of non-limiting example, the predictive analytics module may be configured to operate using the rules and conditions relating to, for example, an external data element such as a threshold data point exhibiting a high pollen count.

In one embodiment, the predictive analytics module may be configured to analyze this external data element to determine whether or not a threshold data point has been met or exceeded. After such a determination is made, the module may then be configured to determine the set of conditions associated with the threshold point being exceeded. The predictive analysis module may be further configured to determine if any of the patient data elements correspond to the set of conditions associated with the threshold point being met or exceeded. The module further configured to determine implications of said rule or condition on qualifying patient profiles based on patient data elements having a positive match or association with the rule or conditions associated with the threshold point being met or exceeded.

As detailed above, the patient profile data relevant to the qualified rule or condition may be determined by the various codes and/or characteristics within the plurality of patient data. Those profiles that have been qualified may then be determined to be relevant to a triggering action associated with the rule and condition. Triggering actions may include, but not be limited to, for example, alerts and or notifications sent to at least one relevant party or entity related to the profile data.

By way of non-limiting example, the predictive analytics module may be configured to be used by a patient, for example, in waypoint navigation from a first destination to a second destination.

In an embodiment, the predictive analytics module, may be configured to trigger alerts to a practitioner and/or patients to ensure the patient takes preventive measures to avoid aggravating or impacting a condition associated with a predetermined threshold being met or exceeded.

By way of a non-limiting example, let's assume that the module has analyzed data and have determined a trend associated with a patient having a diagnosis of Asthma. In this example, the module may have been configured to determine that there exists a high probability that a patient with said diagnosis will have a negative reaction when exposed to air quality with a value above a predetermined threshold.

Based on analysis of external data received by the module, it has been determined that the air quality has met or exceeded the predetermined threshold. That patient is using a navigation application on their mobile device to navigate to a destination. The predictive analytics module may be configured such that it receives information associated with the patient location associated with the patient's mobile device. The patient's mobile device also includes applications which track patient data including, but not limited to, biometric and health information.

The predictive analytics modules may be configured to determine that based on their current route of travel, there is a high probability that the patient will encounter air quality which they should avoid (i.e. fire in the area). As a result, the predictive analytics module can alert the patient, automatically reroute the patient, or suggest that the patient change their route and avoid the affected area.

The aforementioned analyses may be based on, at least in part, a determination of which data elements or predetermined thresholds frequently occur together within an encounter note and/or within an input tier of an encounter note, and/or within a plurality of patient data. In certain embodiments, the predictive analytics module may work in conjunction with favorites module and/or recommendation module. For instance, the predictive analytics module may be configured in a manner that user input is required at each step. In this configuration, the favorites module 125 may determine which data points or data point categories to display within favorites module associated with data entry for the predictive analytics module. For example, the most frequently occurring suggested actions associated with a particular threshold or data element can be determined by the favorites module.

As noted above, the predictive analytics module may be configured to work in conjunction with the recommendation module. For instance, the predictive analytics module may be configured in a manner that user input is required at each step. In this configuration, the recommendation module may provide recommended actions to a user 105 (i.e., avoid this location, reroute your navigation away from this area, avoid outdoors, or another preventive action) based on determinations made by the predictive analytics module.

In other embodiments, all or a portion of this functionality may exist entirely within the predictive analytics module. In yet some other embodiments, the determination and triggering of alerts and suggested preventive actions for the patient is performed automatically or autonomically without the aid of a user.

### a) Doctor Facing: Preemptive Preparedness

As mentioned above, the predictive analytics module further provides methods and systems for making predictions based on an analysis of data accessible to platform 100. Platform 100 may monitor data sources in real-time such that the information available to platform 100 is consistently updated with the most up-to-date information. The predictive analytics module may be configured to correlate patient data against a certain set of rules and conditions. The module may trigger alerts and notifications as a result of analyzing external data relating to the set of conditions and making the determination that certain conditions are present which may affect specific patients.

By way of non-limiting example, the predictive analytics module may be configured to be used by a practitioner, for example, to predict how many walk-ins the practitioner may have and/or how many patients may need help/advice today on certain topics. This will help the practitioner plan/prepare.

### Preemptive Preparedness Analysis

Consistent with the embodiments of the present disclosure, the predictive analytics module may be configured to determine and/or suggest a preemptive preparedness action, a plurality of preemptive preparedness actions, or a series of suggested preemptive preparedness actions to be taken by the practitioner in accordance to a plurality of methods, and through a plurality of systems. Some of those methods and systems include, but are not limited to, for example:

Analysis performed based on a defined set of rules and conditions associated with a plurality of patient data including at least one of: patient profile data, patient tracking data, external data;
Wherein the plurality of patient data further comprises data elements;
Wherein the data elements further comprise at least one of: coded records, patient encounter data, patient monitoring data, external databases, external diagnostic data;
   Analysis performed on data elements based on the rules and conditions;
Wherein rules and conditions include a patient data element;
   Analysis performed on external data to determine whether an element of the external data meets a predetermined threshold;
Wherein the predetermined threshold is associated with the set of conditions;
Wherein the set of conditions may impact one or more patients;
Wherein an positive association match between the external patient data element meeting the predetermined threshold and a patient data element triggers a practitioner alert;
   Analysis performed to determine a correlation between impacted patients and data elements associated with a practitioner
Wherein data elements associated with a practitioner further comprises at least one of: practitioner schedule, practitioner availability, practitioner support staff schedule, practitioner support staff availability, available/stocked supplies, needed supplies, ordered supplies, supplies scheduled for delivery, beds available, beds needed, quarantine rooms available, quarantine rooms needed, or another preparedness action.
Where data elements associated with impacted patients further comprises at least one of: potentially mild symptoms, potentially moderate symptoms, potentially severe symptoms, or another data element associated with impacted patients.
   Analysis performed on the correlation of data to determine a preemptive preparedness action/suggestion/series of actions/suggestions;
Wherein the preemptive preparedness action further comprises alerting the practitioner to prepare for at least one of: a high number of walk-ins, a moderate number of walk-ins, a low number of walk-ins, an average number of walk-ins, or another preemptive preparedness action.
Wherein the preemptive preparedness action further comprises informing the practitioner of the occurrence of at least one of: an outbreak, an epidemic, an extreme crisis, a natural disaster, another preemptive preparedness action.
Wherein the preemptive preparedness action further comprises advising the practitioner to prepare for at least one of the following: a high volume of patient calls, a moderate volume of patient calls, a low volume of patient calls, an average number of patient calls, or another preemptive preparedness action.
Wherein the preemptive preparedness action further comprises suggesting to the practitioner at least one of the following: schedule more practitioner support staff, schedule more on duty practitioners, maintain level of practitioner support staff, reduce level of practitioner support staff, maintain level of available practitioners, reduce level of available practitioners, order more supplies, stock delivered supplies, increase number of beds available, maintain average number of beds available, increase number of quarantine rooms available, maintain number of quarantine rooms available, or another preemptive preparedness action.
   Analysis performed on patient data to determine whether a practitioner qualifies to receive an alert associated with patient;
Wherein analysis further comprises determining whether the necessary practitioner is at least one or more of: a general practitioner, a specialized practitioner, or other type of practitioner.
   Analysis performed on patient data for a plurality of patients to determine whether one or more practitioners qualifies to receive an alert;
   Analysis performed on external data to determine whether the probability of the occurrence an event has reached a predetermined threshold.
Wherein the event impacts patients or a subset of patients
Wherein the event is at least one of: high pollen count, poor air quality, high smog level, high lead level in public water supply, poisonous gas released, high radiation levels, terror attack, fire, flooding, earthquake, tornado, heat wave, drought, or another type of emergent event.

As a consequence of the analysis conducted by the predictive analytics module, a positive correlation between a set of conditions associated with data elements within external data and data elements within patient data determines the level of probability of a possible set of occurrences. Once the probability passes a predetermined threshold value, the predictive analytics module may be further configured to correlate the occurrence/event with the patient data to determine how many patients would be impacted. The predictive analytics module may be further configured to trigger alerts to a practitioner as a consequence of analysis of patient data based on this correlation.

This system allows doctors and health practitioners to prepare to provide certain types of care based on the predictive analysis of patient data. By way of non-limiting example, the predictive analytics module may be further configured to be used by a practitioner, for example, to predict how many walk-ins the practitioner may have based on the high probability of an event occurring. For example, when a smog alert is either issued or predicted by the weather forecast, the module will alert the practitioner to prepare to receive a large number of asthma patients or patients with respiratory problems as walk-ins. In another example, a measles outbreak at a local elementary school may cause the module to suggest to the practitioner to take a number of preemptive preparedness actions (prepare a certain number of quarantine rooms, schedule more practitioner support staff, schedule more available practitioners, request more specialized practitioners, or other type of preemptive preparedness action).

In yet another non-limiting example, the public diagnosis of a popular celebrity with a severe health malady may cause the module to alert the practitioner to prepare to receive a high volume of walk-ins, calls and/or emails relating to the severe health malady. In another possible embodiment, in the event of a terror alert, probable terror attack, or forecasted natural disaster, the module may be configured to suggest a series of preemptive preparedness actions.

The aforementioned analyses may be based on, at least in part, a determination of which data elements, events, preemptive preparedness actions, or predetermined thresholds frequently occur together within an encounter note and/or within an input tier of an encounter note, and/or within a plurality of patient data and/or within external data, and/or within associated data.

In certain embodiments, the predictive analytics module may work in conjunction with favorites module and/or recommendation module. For instance, the predictive analytics module may be configured in a manner that user input is required at each step. In this configuration, the favorites module 125 may determine which data points or data point categories to display within favorites module associated with data entry for the predictive analytics module. For example, the most frequently occurring preemptive preparedness actions associated with a particular threshold or data element can be determined by the favorites module.

As noted above, the predictive analytics module may be configured to work in conjunction with the recommendation module. For instance, the predictive analytics module may be configured in a manner that user input is required at each step. In this configuration, the recommendation module may provide recommended preemptive preparedness actions to a user 105 (i.e., high number of patient calls, high number of walk-ins, low level of necessary supplies, low number of practitioner support staff available, or other type of preemptive preparedness action) based on determinations made by the predictive analytics module.

In other embodiments, all or a portion of this functionality may exist entirely within the predictive analytics module. In yet some other embodiments, the determination and triggering of alerts and suggested preventive actions for the patient is performed automatically or autonomically without the aid of a user.

### III. Platform Operation

FIG. 13A and FIG. 13B are flow charts setting forth the general stages involved in a method 1300 consistent with an embodiment of the disclosure for providing a method for recommending treatment plans, preventive actions, and preparedness actions utilizing a platform 100. Method 1300 may be implemented using a computing device 1500 as described in more detail below with respect to FIG. 15.

Although method 1300 has been described to be performed by computing device 1500, it should be understood that, in some embodiments, different operations may be performed by different networked elements in operative communication with computing device 1500. For example, server 110 and/or computing device 1500 may be employed in the performance of some or all of the stages in method 1300. Moreover, server 110 may be configured much like computing device 1500 and, in some instances, be one and the same embodiment. Similarly, platform apparatus 100 may be employed in the performance of some or all of the stages in method 1300. Apparatus 100 may also be configured much like computing device 1500.

Although method 1400 has been described to be performed by platform 100, it should be understood that computing device 1500 may be used to perform the various stages of method 1400. Furthermore, in some embodiments, different operations may be performed by different networked elements in operative communication with computing device 1500. For example, server 110 may be employed in the performance of some or all of the stages in method 1400. Moreover, server 110may be configured much like computing device 1500. Similarly, platform apparatus 100 may be employed in the performance of some or all of the stages in method 1400. Apparatus 100 may also be configured much like computing device 1500.

Although method 1600 has been described to be performed by platform 100, it should be understood that computing device 1500 may be used to perform the various stages of method 1600. Furthermore, in some embodiments, different operations may be performed by different networked elements in operative communication with computing device 1500. For example, server 110 may be employed in the performance of some or all of the stages in method 1600. Moreover, server 110 may be configured much like computing device 1500. Similarly, platform apparatus 100 may be employed in the performance of some or all of the stages in method 1600. Apparatus 100 may also be configured much like computing device 1500.

Although method 1700 has been described to be performed by platform 100, it should be understood that computing device 1500 may be used to perform the various stages of method 1700. Furthermore, in some embodiments, different operations may be performed by different networked elements in operative communication with computing device 1500. For example, server 110 may be employed in the performance of some or all of the stages in method 1700. Moreover, server 110 may be configured much like computing device 1500. Similarly, platform apparatus 100 may be employed in the performance of some or all of the stages in method 1700. Apparatus 100 may also be configured much like computing device 1500.

Although method 1800 has been described to be performed by platform 100, it should be understood that computing device 1500 may be used to perform the various stages of method 1800. Furthermore, in some embodiments, different operations may be performed by different networked elements in operative communication with computing device 1500. For example, server 110 may be employed in the performance of some or all of the stages in method 1800. Moreover, server 110 may be configured much like computing device 1500. Similarly, platform apparatus 100 may be employed in the performance of some or all of the stages in method 1800. Apparatus 100 may also be configured much like computing device 1500.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1300 will be described in greater detail below.

Method 1300 may begin at starting block 1300 and proceed to stage 1302 where computing device 1500 may provide a first interface for receiving data points associated with a patient encounter. For example, a user interface module may provide an interface for entry of practitioner notes associated with a patient encounter. In general, the first flow chart describes an embodiment of the disclosure in terms of computing device 1500, programming modules 1506 (i.e. Application Modules 1520), and the associated method.

From stage 1304, where computing device 1500 receives data points from a user in an input tier of the first interface, method 1300 may advance to stage 1306 where computing device 1500 may provide a second interface to assist with data entry into the first interface.

Once computing device 1500 provides a second interface to assist with data entry into the first interface in stage 1306, method 1300 may continue to stage 1308 where computing device 1500 may make a plurality of suggested data points available for the user to select and enter data into the first interface.

After computing device 1500 make a plurality of suggested data points available for the user to select and enter data into the first interface in stage 1308, method 1300 may proceed to stage 1310 where computing device 1500 may receive at least one data point or data element from various data sources. For example, data may be received from internal patient data or external data sources.

After computing device 1500 receives at least one data point or data element from various data sources in stage 1310, method 1300 may proceed to stage 1312 where computing device 1500 may analyze the data, data point, or data element.

After computing device 1500 analyzes the data, data point, or data element in stage 1312, method 1300 may proceed to stage 1314 where computing device 1500 may recommend a diagnostic question, treatment, action or other option based on analysis performed.

After computing device 1500 recommends a diagnostic question, treatment, action or other option based on analysis performed in stage 1314, method 1300 may proceed to stage 1316 where computing device 1500 may determine the probable impact of an event occurrence on a patient population. For example, will a probable high pollen count, high smog level, or poor air quality alert adversely affect the patient population associated with the practitioners.

After computing device 1500 determines the probable impact of an event occurrence on a patient population in stage 1316, method 1300 may proceed to stage 1318 where computing device 1500 may analyze the probable impact to determine whether one or more patients or practitioners qualifies to receive an alert for a preventive or preparedness activity. For example, if a patient with asthma is near an area where there is poor air quality, said patient would qualify to receive an alert. Another asthma patient who is a safe determined distance away from the area of poor air quality would not qualify to receive an alert. This patient would qualify if there is a change in their location and they approach a determined radius near the area of poor air quality.

Once computing device 1500 analyzes the probable impact to determine whether one or more patients or practitioners qualifies to receive an alert for a preventive or preparedness activity in stage 1318, method 1300 may then end at stage 1320 or stage 1322 where computing device 1500 may provide a patient with a preventive action, suggestion, recommendation, or other option (1320) or provide a practitioner with a preparedness action, suggestion, recommendation, or other option (1322).

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1400 will be described in greater detail below.

Method 1400 may begin at starting block 1400 and proceed to stage 1402 where computing device 1500 may input data associated with a patient encounter. From stage 1402, where computing device 1500 inputs data associated with a patient encounter, method 1400 may advance to stage 1404 where computing device 1500 may determine at least one patient profile correlating to another patient profile or similar patient profile.

Once computing device 1500 determines at least one patient profile correlating to another patient profile or similar patient profile in stage 1404, method 1400 may continue to stage 1406 where computing device 1500 may analyze data associated with patient data, encounter note, or another patient profile. Once computing device 1500 analyze data associated with patient data, encounter note, or another patient profile in stage 1406, method 1400 may then end at stage 1408 where computing device 1500 provides a recommended treatment, diagnosis, or other data point.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1600 will be described in greater detail below.

Method 1600 may begin at starting block 1600 and proceed to stage 1602 where computing device 1500 may determine whether an occurrence of an event would adversely impact patients within the patient population. For example, will a probable high pollen count, high smog level, or poor air quality alert adversely affect the patient population associated with the practitioner(s).

From stage 1602, where computing device 1500 determines whether an occurrence of an event would adversely impact patients within the patient population, method 1600 may advance to stage 1604 where computing device 1500 may determine whether a probability of an occurrence of an event has reached a predetermined threshold. For example, if there is a seventy-five percent chance of a high pollen count when sixty percent is the predetermined threshold, the probability of a high pollen count has reached a predetermined threshold.

Once computing device 1500 determine whether a probability of an occurrence of an event has reached a predetermined threshold in stage 1604, method 1600 may continue to stage 1606 where computing device 1500 may alert qualified patient(s) or practitioner(s) regarding the high probability of occurrence of the adverse event. For example, if a patient with asthma is near an area where there is poor air quality, said patient would qualify to receive an alert. Another asthma patient who is a safe determined distance away from the area of poor air quality would not qualify to receive an alert. This patient would qualify if there is a change in their location and they approach a determined radius near the area of poor air quality. A practitioner specializing in respiratory care of asthma patients would also qualify for an alert or notification.

After computing device 1500 alert qualified patients or practitioners regarding the high probability of occurrence of the adverse event in stage 1606, method 1600 may proceed to stage 1608 where computing device 1500 may predict a preventive or preparedness action. Once computing device 1500 predicts a preventive or preparedness action in stage 1608, method 1600 may then end at stage 1610 where computing device 1500 may provide the patient(s) or practitioner(s) with a preventive or preparedness action. For example, if a patient with asthma is near an area where there is poor air quality, said patient would receive a preventive action such as but not limited to, “avoid the area” or “reroute your travel around the area.” A practitioner specializing in respiratory care of asthma patients would also receive a preparedness action such as but not limited to, “prepare for a high number of walk-ins” or “order more asthma inhalers and respiratory treatment supplies.”

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1700 will be described in greater detail below.

Method 1700 may begin at starting block 1700 and proceed to stage 1702 where computing device 1500 may receive at least one data element. From stage 1702, where computing device 1500 receives at least one data element, method 1700 may advance to stage 1704 where computing device 1500 may analyze the data element. For example, the favorites module, recommendation module, patient tracking module, or predictive analytics module may analyze the data element. Once computing device 1500 analyzes the data element in stage 1704, method 1700 may then end at stage 1706 where computing device 1500 may make a recommendation based on the analysis. For example, the recommendation module may recommend a diagnostic question, recommended treatment, or recommended action.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1800 will be described in greater detail below.

Method 1800 may begin at starting block 1800 and proceed to stage 1802 where computing device 1500 may receive data points from a patient entered by a patient, practitioner, or user. For example, a user interface module may provide an interface for entry of practitioner notes associated with a patient encounter. From stage 1802, where computing device 1500 receives data points from a user in an input tier, method 1800 may advance to stage 1804 where computing device 1500 may receive treatment notes for a patient.

Once computing device 1500 receives treatment notes for a patient in stage 1804, method 1800 may continue to stage 1806 where computing device 1500 may specify patient data points in a first tier relating to reasons for visit or chief complaints. After computing device 1500 specifies patient data points in a first tier relating to reasons for visit or chief complaints in stage 1806, method 1800 may proceed to stage 1808 where computing device 1500 may specify patient data points in a second tier relating to history reasons for visit or history of present illness.

After computing device 1500 specifies patient data points in a second tier relating to history reasons for visit or history of present illness in stage 1808, method 1800 may proceed to stage 1810 where computing device 1500 specify a body system in a third tier relating to the first tier or second tier. After computing device 1500 specifies a body system in a third tier relating to the first tier or second tier in stage 1810, method 1800 may proceed to stage 1812 where computing device 1500 may specify a fourth tier a condition associated with the body system specified in the third tier.

After computing device 1500 specifies a fourth tier a condition associated with the body system specified in the third tier in stage 1812, method 1800 may proceed to stage 1814 where computing device 1500 may specify a treatment plan in a fifth tier. Once computing device 1500 specifies a treatment plan in stage 1814, method 1800 may then end at stage 1816 where computing device 1500 may specify a treatment in a sixth tier.

### IV. Computing Device Architecture

The platform 100 may be embodied as, for example, but not be limited to, a website, a web application, a desktop application, and a mobile application compatible with a computing device. The computing device may comprise, but not be limited to, a desktop computer, laptop, a tablet, or mobile telecommunications device (105A, 145A). Moreover, the platform 100 may be hosted on a centralized server, such as, for example, a cloud computing service. Although methods 1300, 1400, 1600, 1700, and 1800 have been described to be performed by a computing device 1500, it should be understood that, in some embodiments, different operations may be performed by different networked elements in operative communication with computing device 1500.

Embodiments of the present disclosure may comprise a system having a memory storage and a processing unit. The processing unit coupled to the memory storage, wherein the processing unit is configured to perform the stages of methods 1300, 1400, 1600, 1700, and 1800.

FIG. 15 is a block diagram of a system including computing device 1500. Consistent with an embodiment of the disclosure, the aforementioned memory storage and processing unit may be implemented in a computing device, such as computing device 1500 of FIG. 15. Any suitable combination of hardware, software, or firmware may be used to implement the memory storage and processing unit. For example, the memory storage and processing unit may be implemented with computing device 1500 or any of other computing devices 1518, in combination with computing device 1500. The aforementioned system, device, and processors are examples and other systems, devices, and processors may comprise the aforementioned memory storage and processing unit, consistent with embodiments of the disclosure.

With reference to FIG. 15, a system consistent with an embodiment of the disclosure may include a computing device, such as computing device 1500. In a basic configuration, computing device 1500 may include at least one processing unit 1502 and a system memory 1504. Depending on the configuration and type of computing device, system memory 1504 may comprise, but is not limited to, volatile (e.g. random access memory (RAM)), non-volatile (e.g. read-only memory (ROM)), flash memory, or any combination. System memory 1504 may include operating system 1505, one or more programming modules 1506, and may include a program data 1507. Operating system 1505, for example, may be suitable for controlling computing device 1500's operation. In one embodiment, programming modules 1506 may include a user interface module, favorites module, recommendation module, predictive analytics module, preventive care module, preparedness module, and communication module. Furthermore, embodiments of the disclosure may be practiced in conjunction with a graphics library, other operating systems, or any other application program and is not limited to any particular application or system. This basic configuration is illustrated in FIG. 15 by those components within a dashed line 1508.

Computing device 1500 may have additional features or functionality. For example, computing device 1500 may also include additional data storage devices (removable and/or non-removable) such as, for example, magnetic disks, optical disks, or tape. Such additional storage is illustrated in FIG. 15 by a removable storage 1509 and a non-removable storage 1510. Computer storage media may include volatile and nonvolatile, removable and non-removable media implemented in any method or technology for storage of information, such as computer readable instructions, data structures, program modules, or other data. System memory 1504, removable storage 1509, and non-removable storage 1510 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1500. Any such computer storage media may be part of device 1500. Computing device 1500 may also have input device(s) 1512 such as a keyboard, a mouse, a pen, a sound input device, a touch input device, etc. Output device(s) 1514 such as a display, speakers, a printer, etc. may also be included. The aforementioned devices are examples and others may be used.

Computing device 1500 may also contain a communication connection 1516 that may allow device 1500 to communicate with other computing devices 1518, such as over a network in a distributed computing environment, for example, an intranet or the Internet. Communication connection 1516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1504, including operating system 1505. While executing on processing unit 1502, programming modules 1506 (e.g., a user interface module, favorites module, recommendation module, predictive analytics module, preventive care module, preparedness module, and communication module) may perform processes including, for example, one or more of method 1300's stages as described above. The aforementioned process is an example, and processing unit 1502 may perform other processes. Other programming modules that may be used in accordance with embodiments of the present disclosure may include electronic mail and contacts applications, word processing applications, spreadsheet applications, database applications, slide presentation applications, drawing or computer-aided application programs, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and quantum computing elements. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

All rights including copyrights in the code included herein are vested in and the property of the Applicant. The Applicant retains and reserves all rights in the code included herein, and grants permission to reproduce the material only in connection with reproduction of the granted patent and for no other purpose.

### Other Embodiments

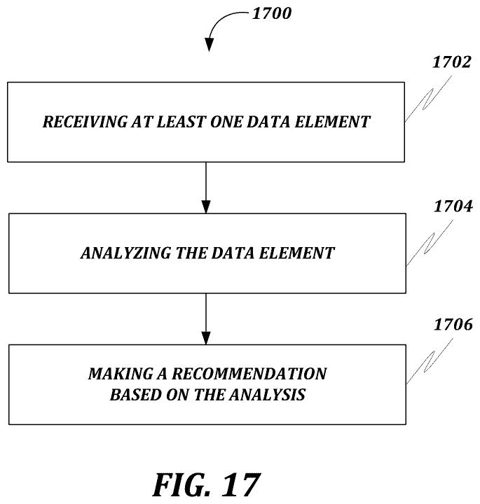

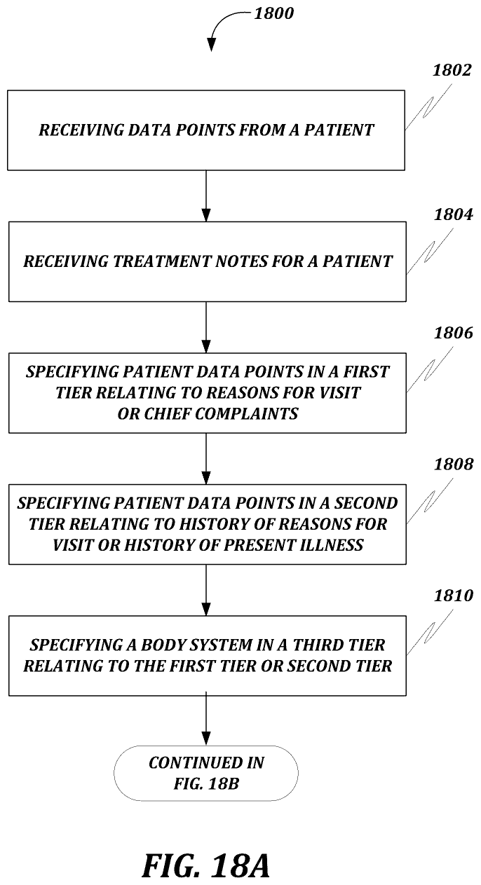

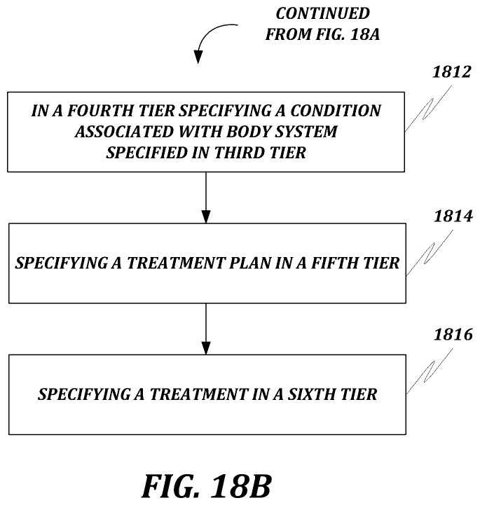

The aforementioned modules may be used in various combinations with one another. The modules can also be used together as separate elements of a system working together but not contained within the same component. In other embodiments, the modules can be contained within the same component or used as an element or elements of a system. In yet other embodiments, the functionality for any of the aforementioned modules can be embodied in one individual module alone or collectively in a portion of the modules. The modules can be embodied as software, hardware or a combination thereof. As described in the present disclosure, the modules can also be collectively embodied in one device, server, computer readable medium, or hardware element. FIGS. 17 and 18 illustrate various additional methods available to be implemented consistent with embodiments of the present disclosure.

### Other Capabilities

The modules may further be configured to utilize existing technologies known to one of ordinary skill in the art. Mobile devices, laptops, cameras, smartphones, computers, sensors, sensory devices, personal sensory devices, microphones, electronic devices, and other devices may be utilized with any of the aforementioned modules. By way of non-limiting example, during a patient encounter, a camera, smartphone, mobile device, computer, or electronic device can be used to capture audio, video, photographs, or other media and/or multimedia content associated with the encounter. In another non-limiting example, alerts generated using the predictive analytics module may provide a patient with a video message from his or her practitioner with delivery of the preventive action. By way of another non-limiting example, alerts generated using the predictive analytics module may provide a practitioner with a video snippet from the news report, a snippet from a news article, a photograph of an affected patient, or other media with delivery of the preemptive preparedness action. This information can be stored for use with the server, external data or other components of the system.

### V. Aspects

The following disclose various Aspects of the present disclosure. The various Aspects are not to be construed as patent claims unless the language of the Aspect appears as a patent claim. The Aspects describe various non-limiting embodiments of the present disclosure.

### Aspects of the User Interface Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. An input system comprising, but not limited to, at least two stages of assisted data entry, the input system comprising: a first stage of input for receiving data points from a patient; a second stage of input for receiving treatment notes for the patient, and wherein the first stage of input comprises a plurality of tiers for specifying the data points for the patient, the plurality of tiers comprising at least one of the following: a first tier for specifying at least one of the following: reasons for visit/chief complaints; a second tier for specifying at least one of the following: History of Reason for visit/History of Present Illness (HPI); a third tier for specifying a body system associated with at least one of the following: the first tier and the second tier; a fourth tier for specifying a condition associated with the body system specified in the third tier; wherein the second stage of input comprises a plurality of tiers for specifying the treatment notes for the patient, the plurality of tiers comprising at least one of the following: a fifth tier for specifying a treatment plan; and a sixth tier for specifying a treatment in accordance to the treatment plan.
Aspect 2. The input system of Aspect 1 wherein the first stage of input and the second stage of input are compiled into a patient encounter note.
Aspect 3. The input system of Aspect 1 wherein a completion of the patient encounter note is facilitated, at least in part, by at least one of the following: a favorites module; a recommendation module; and an analytics module.

### Aspects of the Favorites Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. An input system comprising, but not limited to, at least two stages of assisted data entry, the input system comprising: providing a first interface for receiving data points associated with a patient encounter, wherein the first interface comprises: an input tier for receiving data points from a user; and providing a second interface for assistant with an entry of the data points into the first interface, wherein the second interface comprises: a plurality of suggested data points made available for user selection, wherein at least one selected suggested data point is configured to populate a currently selected input tier of the first interface.
Aspect 2. The input system of Aspect 1, wherein the first interface further comprises a sub-input tier for categorizing the received data points from the user within the input tier.
Aspect 3. The input system of Aspect 1, wherein the second interface further comprises a plurality of suggested data point categories configured to populate a currently selected input tier of the first interface.
Aspect 4. The input system of Aspect 1, wherein the plurality of suggested data point categories are configured to populate a/the currently selected input tier of the first interface as a sub-input tier.
Aspect 5. The input system of Aspect 4, wherein the sub-input tier is configured to categorize data point received within the input tier of the first interface.
Aspect 6. The input system of Aspect 4, wherein the suggested data points are tailored to a currently selected input tier in the first interface.
Aspect 7. The input system of Aspect 6, wherein the suggested data point categories are tailored to a currently selected input tier in the first interface.
Aspect 8. The input system of Aspect 4, wherein the suggested data points are based on at least one of the following: analysis performed on previous inputs for a current patient encounter; analysis performed on previous patient encounters for the same patient; and analysis performed on previous patient encounters for a plurality of patients.
Aspect 9. The input system of Aspect 8, Wherein the analysis performed on previous inputs for a current patient encounter comprises analyzing inputs made in a current input tier.
Aspect 10. The input system of Aspect 8, wherein the analysis performed on previous inputs for a current patient encounter comprises analyzing completed input tiers for the current patient encounter.
Aspect 11. The input system of Aspect 10, wherein the plurality of patient encounter data used for the analysis is accessed through an internal dataset.
Aspect 12. The input system of Aspect 10, wherein the plurality of patient encounter data used for the analysis is accessed through an external dataset.
Aspect 13. The input system of Aspect 10, wherein the analysis is configured to determine which data points frequently occur together within the input tier.
Aspect 14. The input system of Aspect 10, wherein the analysis is configured to determine a frequency with which the data points were previously used within the input tier.
Aspect 15. The input system of Aspect 10, wherein the analysis is configured to determine which data points frequently occur together across more than one input tier.
Aspect 16. The input system of Aspect 10, wherein the analysis is configured to determine a frequency with which the data points occur together across more than one input tier.
Aspect 17. The input system of Aspect 10, wherein the analysis is configured to determine which data point categories frequently occur together within the input tier.
Aspect 18. The input system of Aspect 10, wherein the analysis is configured to determine a frequency with which the data point categories were previously used within the input tier.
Aspect 19. The input system of Aspect 10, wherein the analysis is configured to determine which data point categories frequently occur together across more than one input tier.
Aspect 20. The input system of Aspect 10, wherein the analysis is configured to determine a frequency with which the data point categories occur together across more than one input tier.
Aspect 21. The input system of Aspect 10, wherein the second interface further comprises a graphical user interface (GUI) element used to indicate a frequency with which each of the suggested data points has previously been used.
Aspect 22. The input system of Aspect 21, wherein the GUI element is configured to accompany a display for each of the suggested data points.
Aspect 23. The input system of Aspect 21, wherein a color of the GUI element is configured to convey an occurrence frequency of the accompanying suggested data point.
Aspect 24. The input system of Aspect 21, wherein an intensity of the color of the GUI element is configured to convey an occurrence frequency of the accompanying suggested data point.

### Aspects of Recommendation Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. A method for provided improved patient diagnostics, the method comprising: receiving at least one data point in a patient encounter note during a patient diagnostic process; establishing a patient profile based on the at least one data point in the patient encounter note; analyzing the patient profile; recommending, based on the analysis, at least one of the following: a diagnostic question, a recommended treatment, and a recommended action.
Aspect 2. The method of Aspect 1 wherein the patient diagnostic process is configured to guide a user through inputting data associated with a patient encounter, and wherein a recommendation is provided in furtherance of completing the patient diagnostic process.
Aspect 3. The method of Aspect 1 wherein analyzing the patient profile comprises detecting a correlation in data point entries within the patient encounter note.
Aspect 4. The method of Aspect 1 wherein analyzing the patient profile comprises detecting a correlation in data point entries with other data point entries in other patient encounter notes.
Aspect 5. The method of Aspect 1 wherein analyzing further comprises: determining at least one patient profile similar to the patient profile associated with the patient encounter note; analyzing data points within at least one patient encounter note associated with the at least one similar patient profile; and determining a recommendation based on the analysis of the patient encounter note and the at least one patient encounter note associated with the at least one similar patient profile.
Aspect 6. The method of Aspect 1 wherein analyzing data points within the at least one patient encounter note associated with the at least one similar patient profile comprises:
Time Based
Location Based
Gender Based
Age Based
Medical History Based
Family History Based
Social History Based
Surgical History Based
Based on Demographic Data
   Aspect 7. The method of Aspect 1 wherein analyzing data points within the at least one patient encounter note associated with the at least one similar patient profile comprises: previous encounters for each patient profile; previous diagnosis for each patient profile; and previous treatments for each patient profile.
   Aspect 8. The method of Aspect 1 wherein the suggested data points are based on at least one of the following: analyzing previous inputs for a current patient encounter; and analyzing performed on previous patient encounters for the same patient; and analyzing performed on previous patient encounters for a plurality of patients.
   Aspect 9. The method of Aspect 1 wherein analyzing data points within the at least one patient encounter note associated with the at least one similar patient profile comprises: Reasons for visit/chief complaints for each encounter note in each patient profile; and History of Reason for visit/History of Present Illness (HPI) for each encounter note in each patient profile.
   Aspect 10. The method of Aspect 1 wherein analyzing the patient profile comprises accessing an external database.
   Aspect 11. The method of Aspect 1 wherein accessing the external database comprises employing an application programming interface (API).
   Aspect 12. The method of Aspect 1 wherein accessing the external database comprises accessing at least one of the following: Medical Encyclopedias, and WHO databases.
   Aspect 13. The method of Aspect 1 wherein analyzing the patient profile comprises accessing environmental sensor data.
   Aspect 14. The method of Aspect 12 wherein accessing environmental sensor data comprises accessing data obtained from at least one of the following: weather sensors, air quality sensors, and water sensors.
   Aspect 15. The method of Aspect 1 wherein recommending, based on the analysis, comprises at least one of the following: displaying question to ask; receiving validation that question has been asked; providing data point entry for inputting an answer; and recording the data point entry in the encounter note.
   Aspect 16. The method of Aspect 15 wherein providing the data point entry comprises updating a display of suggested data point entries in a favorites module.
   Aspect 17. The method of Aspect 5 wherein recommending, based on the analysis, comprises providing at least one treatment option for entry into the encounter note.
   Aspect 18. The method of Aspect 17 wherein providing the at least one treatment option for entry into the encounter note comprises updating a display of suggested data point entries in a favorites module.
   Aspect 19. The method of Aspect 1 Wherein recommending, based on the analysis, comprises at least one of the following: displaying a recommended action to execute; receiving validation that action has been executed; providing data point entry for updating the encounter note; and recording the data point entry in the encounter note upon selection of the data point entry.
   Aspect 20. The method of Aspect 19 wherein providing the data point entry comprises updating a display of suggested data point entries in a favorites module.

### Aspects of Preventive Care Sub-Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. A method for providing preventive actions to patients, the method comprising: receiving at least one data element associated with a plurality of patient data; analyzing the patient data; determining, based on the analysis, whether a set of rules and conditions may cause an adverse effect on a patient; predicting, based on the analysis, at least one of the following: a preventive suggestion, and a preventive action.
Aspect 2. The method of Aspect 1 wherein the data elements further comprise at least one of: coded records, patient encounter data, patient monitoring data, external databases, and external diagnostic data.
Aspect 3. The method of Aspect 1 wherein analyzing data elements is based on the rules and conditions.
Aspect 4. The method of Aspect 1 wherein analyzing further comprises: determining whether a data element meets a predetermined threshold.
Aspect 5. The method of Aspect 4 wherein the predetermined threshold is associated with the set of conditions.
Aspect 6. The method of Aspect 3 wherein determining further comprises: determining whether there is a high probability that a known set of conditions will adversely impact one or more patients.
Aspect 7. The method of Aspect 6 wherein analyzing further comprises: detecting a correlation between an external patient data element meeting the predetermined threshold and a patient data element.
Aspect 8. The method of Aspect 6 wherein determining further comprises: detecting a correlation between an external patient data element meeting the predetermined threshold and a patient data element.
Aspect 9. The method of Aspect 6 wherein determining further comprises: a positive correlation between an external patient data element meeting the predetermined threshold and a patient data element triggers an alert.
Aspect 10. The method of Aspect 7 wherein analyzing further comprises determining whether patients should receive an alert based on patient profile data.
Aspect 11. The method of Aspect 1 wherein analyzing the patient profile comprises detecting a correlation in data point entries with other data point entries in other patient encounter notes.
Aspect 12. The method of Aspect 1 wherein analyzing further comprises: determining at least one patient profile similar to the patient profile associated with the set of conditions; analyzing data points within at least one patient data element associated with the at least one determined set of conditions; and determining a preventive action based on the analysis of the patient data element and the at least one external data element associated with the at least one condition.

### Aspects of the Preparedness Sub-Module

The following disclose various Aspects of the present disclosure. The various Aspects are not to be construed as patent claims unless the language of the Aspect appears as a patent claim. The Aspects describe various non-limiting embodiments of the present disclosure.

Aspect 1. A method for providing preemptive preparedness actions to practitioners, the method comprising: receiving at least one data element associated with a plurality of patient data; analyzing the data element; determining, based on the analysis, whether an occurrence of an event may cause an adverse effect on a patient; predicting, based on the analysis, at least one of the following: a preemptive preparedness suggestion, and a preemptive preparedness action; providing the practitioner with at least one of the following: the preemptive preparedness suggestion, and the preemptive preparedness action.
Aspect 2. A method for providing preemptive preparedness actions to practitioners, the method comprising: receiving at least one data element associated with at least one of: a plurality of patient data, a plurality of external data, a plurality of patient profile data, a plurality of encounter notes, patient tracking data, other data; analyzing the data element; determining, based on the analysis, whether an occurrence of an event may cause an adverse effect on a patient; predicting, based on the analysis, at least one of the following: a preemptive preparedness suggestion, and a preemptive preparedness action; providing the practitioner with at least one of the following: a preemptive preparedness suggestion, and a preemptive preparedness action.
Aspect 3. The method of Aspect 1 or Aspect 2 further comprising: wherein determining further comprises whether a probability of the occurrence of an event has met or exceeded a predetermined threshold.
Aspect 4. The method of Aspect 3 wherein the predetermined threshold is associated with the set of conditions.
Aspect 5. The method of Aspect 3 wherein determining further comprises: determining whether there is a high probability that a known set of conditions will adversely impact one or more patients.
Aspect 6. The method of Aspect 3 wherein analyzing further comprises: detecting a correlation between an external patient data element meeting the predetermined threshold, a patient data element, and data elements associated with a practitioner.
Aspect 7. The method of Aspect 3 wherein determining further comprises: detecting a correlation between impacted patients and data elements associated with a practitioner.
Aspect 8. The method of Aspect 3 wherein determining further comprises: a positive correlation between an external patient data element meeting the predetermined threshold and a patient data element triggers a practitioner alert.
Aspect 9. The method of Aspect 3 wherein analyzing further comprises: determining whether practitioners should receive an alert based on patient profile data.
Aspect 10. The method of Aspect 3 wherein analyzing the patient profile comprises: detecting a correlation in data point entries with other data point entries in other patient encounter notes.
Aspect 11. The method of Aspect 3 wherein analyzing further comprises: determining at least one patient profile similar to the patient profile associated with the set of conditions; analyzing data points within at least one patient data element associated with the at least one determined set of conditions; and determining a preemptive preparedness action based on the analysis of the patient data element and the at least one external data element associated with the at least one condition.
Aspect 12. The method of Aspect 3 wherein analyzing further comprises: determining at least one patient profile similar to the patient profile associated with the set of conditions; analyzing data points within at least one patient data element associated with the at least one determined set of conditions; and determining a preemptive preparedness action based on the analysis of the patient data element and the at least one external data element associated with the at least one condition.
Aspect 13. The method of Aspect 3 wherein data elements associated with a practitioner further comprises at least one of: practitioner schedule, practitioner availability, practitioner support staff schedule, practitioner support staff availability, available/stocked supplies, needed supplies, ordered supplies, supplies scheduled for delivery, beds available, beds needed, quarantine rooms available, quarantine rooms needed, or other data element associated with the practitioner.
Aspect 14. The method of Aspect 3 wherein data elements associated with impacted patients further comprises at least one of: potentially mild symptoms, potentially moderate symptoms, potentially severe symptoms, or other data element associated with impacted patients.
Aspect 15. The method of Aspect 3 wherein the analysis, based on the correlation of data, further comprises determining at least one of: a preemptive preparedness action, a preemptive preparedness suggestion, a series of preemptive preparedness actions, a series of preemptive preparedness suggestions.
Aspect 16. The method of Aspect 3 wherein the preemptive preparedness action further comprises alerting the practitioner to prepare for at least one of: a high number of walk-ins, a moderate number of walk-ins, a low number of walk-ins, an average number of walk-ins, or other preemptive preparedness action.
Aspect 17. The method of Aspect 3 wherein the preemptive preparedness action further comprises informing the practitioner of the occurrence of at least one of: an outbreak, an epidemic, an extreme crisis, a natural disaster, or other emergent event.
Aspect 18. The method of Aspect 3 wherein the preemptive preparedness action further comprises advising the practitioner to prepare for at least one of the following: a high volume of patient calls, a moderate volume of patient calls, a low volume of patient calls, an average number of patient calls, or other preemptive preparedness action.
Aspect 19. The method of Aspect 3 wherein the preemptive preparedness action further comprises suggesting to the practitioner at least one of the following: schedule more practitioner support staff, schedule more on duty practitioners, maintain level of practitioner support staff, reduce level of practitioner support staff, maintain level of available practitioners, reduce level of available practitioners, order more supplies, stock delivered supplies, increase number of beds available, maintain average number of beds available, increase number of quarantine rooms available, maintain number of quarantine rooms available, or other preemptive preparedness action.
Aspect 20. A method for providing preemptive preparedness actions to practitioners, the method comprising: receiving at least one data element associated with at least one of: a plurality of patient data, a plurality of external data, a plurality of patient profile data, a plurality of encounter notes, a plurality of patient tracking data, other data; analyzing the data element, determining whether a practitioner qualifies to receive an alert associated with patient, providing an alert to the practitioner.
Aspect 21. The method of Aspect 20 wherein determining further comprises determining whether the practitioner is at least one or more of: a general practitioner, a specialized practitioner.
Aspect 22. A method for providing preemptive preparedness actions to practitioners, the method comprising: receiving at least one data element associated with at least one of: a plurality of patient data, a plurality of external data, a plurality of patient profile data, a plurality of encounter notes, a plurality of patient tracking data, other data; analyzing patient data for a plurality of patients; determining whether one or more practitioners qualifies to receive an alert; providing an alert to the one or more practitioners.
Aspect 23. The method of Aspect 22 wherein analyzing an external data element further comprises determining whether the probability of the occurrence an event has reached a predetermined threshold.
Aspect 24. The method of Aspect 23 wherein the occurrence of an event impacts patients or a subset of patients.
Aspect 25. The method of Aspect 23 wherein the occurrence of an event is at least one of: high pollen count, poor air quality, high smog level, high lead level in public water supply, poisonous gas released, high radiation levels, terror attack, fire, flooding, earthquake, tornado, heat wave, drought, or other emergent event.

### VI. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A computer readable medium comprising, but not limited to, at least one of the following:
an input methodology comprising, but not limited to, at least two stages of assisted data entry; and
an input assistance methodology comprising, but not limited to, at least one of the following:
a favorites module,
a recommendation module,
an analytics module; and
a server in communication with the input methodology and the input assistance methodology.

2. The computer readable medium of claim 1 further comprising:
an improved patient diagnostics method comprising:
receiving at least one data element associated with at least one of the following:
a plurality of patient data, and
at least one data point in a patient encounter note during a patient diagnostic process; and
analyzing the data element.

3. The computer readable medium of claim 1,
the input assistance methodology further comprising:
recommending, based on an analysis, at least one of the following:
a diagnostic question,
a recommended treatment, and
a recommended action.

4. The computer readable medium of claim 1 further comprising:
an analytics method comprising:
determining, based on an analysis, whether an occurrence of an event may cause an adverse effect on a patient;
predicting, based on an analysis, at least one of the following:
a preventive suggestion,
a preventive action,
a preemptive preparedness suggestion, and
a preemptive preparedness action.

5. The computer readable medium of claim 4 further comprising:
an action generating method comprising:
providing a patient or a practitioner with at least one of the following:
the preventive suggestion,
the preventive action,
the preemptive preparedness suggestion, and
the preemptive preparedness action.

6. An input system comprising, but not limited to, at least two stages of assisted data entry, the input system comprising:
a first stage of input for receiving data points from a patient;
a second stage of input for receiving treatment notes for the patient, and
wherein the first stage of input comprises a plurality of tiers for specifying the data points for the patient, the plurality of tiers comprising at least one of the following:
a first tier for specifying at least one of the following:
reasons for visit,
chief complaints;
a second tier for specifying at least one of the following:
History of Reason for visit,
History of Present Illness (HPI);
a third tier for specifying a body system associated with at least one of the following:
the first tier, and
the second tier;
a fourth tier for specifying a condition associated with the body system specified in the third tier;
wherein the second stage of input comprises a plurality of tiers for specifying the treatment notes for the patient, the plurality of tiers comprising at least one of the following:
a fifth tier for specifying a treatment plan; and
a sixth tier for specifying a treatment in accordance to the treatment plan.

7. The input system of claim 6, further comprising:
wherein the first stage of input and the second stage of input are compiled into a patient encounter note.

8. The input system of claim 7, further comprising:
wherein a completion of the patient encounter note is facilitated, at least in part, by at least one of the following:
a favorites module;
a recommendation module; and
an analytics module.

9. The input system of claim 6, wherein the input system further comprises:
providing a first interface for receiving data points associated with a patient encounter, wherein the first interface comprises:
an input tier for receiving data points from a user; and
providing a second interface for assistant with an entry of the data points into the first interface, wherein the second interface comprises:
a plurality of suggested data points made available for user selection,
wherein at least one selected suggested data point is configured to populate a currently selected input tier of the first interface.

10. The input system of claim 9, wherein the suggested data points are based on at least one of the following:
analyzing previous inputs for a current patient encounter; and
analyzing performed on previous patient encounters for the same patient; and
analyzing performed on previous patient encounters for a plurality of patients.

11. The input system of claim 6, wherein an analysis is configured to determine at least one of the following:
which data points frequently occur together within at least one of:
the first tier, the second tier, the third tier, the fourth tier, the fifth tier, the sixth tier; and
a frequency with which the data points were previously used within at least one of:
the first tier, the second tier, the third tier, the fourth tier, the fifth tier, the sixth tier; and
which data points frequently occur together across more than at least one tier. of:
the first tier, the second tier, the third tier, the fourth tier, the fifth tier, the sixth tier; and
which the data points occur together across more than within at least one of:
the first tier, the second tier, the third tier, the fourth tier, the fifth tier, the sixth tier.

12. A method comprising:
an input method comprising:
providing a first interface for receiving data points associated with a patient encounter, wherein the first interface comprises:
an input tier for receiving the data points from a user; and
providing a second interface for assisting with an entry of the data points into the first interface, wherein the second interface comprises:
a plurality of suggested data points made available for user selection, wherein at least one selected suggested data point is configured to populate a currently selected input tier of the first interface; and
an improved patient diagnostics method comprising:
receiving at least one data element associated with at least one of the following:
a plurality of patient data, and
at least one data point in a patient encounter note during a patient diagnostic process;
analyzing the data element;
an input assistance method comprising:
recommending, based on the analysis, at least one of the following:
a diagnostic question,
a recommended treatment, and
a recommended action
an analytics method comprising:
determining, based on an analysis, whether an occurrence of an event may cause an adverse effect on a patient;
predicting, based on the analysis, at least one of the following:
a preventive suggestion,
a preventive action,
a preemptive preparedness suggestion, and
a preemptive preparedness action;
an action generating method comprising:
providing the patient or practitioner with at least one of the following:
the preventive suggestion,
the preventive action,
the preemptive preparedness suggestion, and
the preemptive preparedness action.

13. The method of claim 12 further comprising:
wherein the patient diagnostic process is configured to guide a user through inputting data associated with a patient encounter, and
wherein a recommendation is provided in furtherance of completing the patient diagnostic process.

14. The method of claim 12 wherein analyzing further comprises:
determining at least one patient profile similar to a patient profile associated with the patient encounter note;
analyzing data points within at least one patient encounter note associated with the at least one similar patient profile; and
determining a recommendation based on the analysis of the patient encounter note and the at least one patient encounter note associated with the at least one similar patient profile.

15. The method of claim 12 further comprising:
wherein analyzing data points within the at least one patient encounter note associated with the at least one similar patient profile comprises analyzing at least one of the following:
previous encounters for a patient profile;
previous diagnosis for the patient profile; and
previous treatments for the patient profile.

16. The method of claim 12 further comprising:
receiving at least one data element associated with at least one of:
a plurality of patient data,
a plurality of external data,
a plurality of patient profile data,
a plurality of encounter notes,
a plurality of patient tracking data,
other data;
analyzing patient data for a plurality of patients; and
determining whether one or more practitioners qualifies to receive an alert; and
providing the alert to the one or more practitioners.

17. The method of claim 12 further comprising:
determining whether one or more patients qualifies to receive an alert; and
providing the alert to the one or more patients.

18. The method of claim 12, wherein analyzing further comprises:
determining at least one patient profile similar to a patient profile associated with a set of conditions;
analyzing data points within at least one patient data element associated with at least one determined set of conditions; and
determining a preemptive preparedness action based on the analysis of the at least one patient data element and the at least one external data element associated with at least one condition.

19. The method of claim 18, further comprising:
wherein determining further comprises determining whether there is a high probability that a known set of conditions will adversely impact one or more patients;
wherein analyzing further comprises determining whether a probability of an occurrence an event has reached a predetermined threshold; and
wherein determining further comprises a positive correlation between an external patient data element meeting the predetermined threshold and a patient data element triggers an alert.

20. The method of claim 19, further comprising:
wherein the occurrence of an event is at least one of: high pollen count, poor air quality, high smog level, high lead level in public water supply, poisonous gas released, high radiation levels, terror attack, fire, flooding, earthquake, tornado, heat wave, drought, or other emergent event.
